(19) United States
(12) Patent Application Publication
(10) Pub. No.: US 2025/0378913 A1
(43) Pub. Date: Dec. 11, 2025

(54) METHODS AND SYSTEMS FOR MODELING BIOLOGICAL SYSTEMS, AND APPLICATIONS THEREOF

(71) Applicant: Syntensor Incorporated, Burlington, VT (US)

(72) Inventors: Clayton M. Rabideau, Burlington, VT (US); Archis S. Joglekar, Burlington, VT (US); Nicole Gabrielle Griffin, Burlington, VT (US)

(21) Appl. No.: 18/874,342

(22) PCT Filed: Jun. 15, 2023
(86) PCT No.: PCT/US2023/068551

§ 371 Date: Dec. 12, 2024

Related U.S. Application Data
(60) Provisional application No. 63/352,586, filed on Jun. 15, 2022.

(51) Int. Cl. G16B 40/00; G16B 45/00

(52) U.S. Cl. CPC G16B 40/00; G16B 45/00

(57) ABSTRACT

The present disclosure provides methods and systems for modeling cellular behavior. A method for generating a model of a biological system may include obtaining sample data including records derived from samples of the biological system. The records may indicate the presence, absence, and/or expression levels of entities in respective samples of the biological system. The method may further include dividing the sample data into a training set and a validation set, providing biological system data as input to a machine learning model to initialize the model, training the model to model dynamic behavior of the biological system based on the training set, and validating the trained model using the validation set. The biological system data may include a bipartite graph representing the biological system and structured as an optimal control loop.

### CROSS REFERENCE TO RELATED APPLICATION(S)

This application claims the benefit of and priority to U.S. Provisional Patent Application No. 63/352,586 filed Jun. 15, 2022, the entire disclosure of which is hereby incorporated by reference herein in its entirety for all purposes.

### FIELD OF TECHNOLOGY

The present disclosure relates generally to the fields of machine learning and artificial intelligence, computational biology, and bioinformatics, and more specifically to methods and systems for modeling biological systems.

### BACKGROUND

Many scientific disciplines and fields of engineering have simulators that enable rapid iteration and testing of complex interactions in virtual systems and models rather than in physical experiments. Electrical engineering has electrical rule check (ERC)/design rule check (DRC), computer engineering has unit testing/continuous integration (CI)/continuous deployment (CD), aerospace engineering has Navier-Stokes and Bernoulli-based fluid dynamics simulations, and integrated circuits have electronic design automation (EDA) tools such as simulation program with integrated circuit emphasis (SPICE). Each of these fields and the resulting improvements in their downstream output rely heavily on systems simulators to attain and maintain their current level of complexity.

In contrast to physical systems such as electronic circuits, the governing equations of biological systems are generally unknown. This lack of governing equations prevents (or substantially inhibits) first-principles modeling of the dynamics between a drug, disease, and a cell or tissue. Often, heuristic approaches to modeling are taken which can and do mislead, and when implemented downstream, yield undesirable outcomes. Thus, methods and systems for correctly modeling biological systems are needed.

### SUMMARY OF THE INVENTION

According to an aspect of the present disclosure, a method for generating a model of a biological system is provided. The method comprises obtaining biological system data including architectural data (GL1) and class data, wherein the architectural data represent a bipartite graph representing a biological system, wherein (i) the graph includes a first plurality of entity nodes representing a plurality of entities included in the biological system, a second plurality of interaction nodes representing a plurality of interactions between respective subsets of the entities, and a plurality of directed edges connecting a plurality of node pairs, each node pair including a respective first node representing an entity of the plurality of entities and a respective second node representing an interaction of the plurality of interactions, (ii) the graph is structured as a closed-loop control system, and (iii) the architectural data (GL1) include an initial architectural encoding including a first plurality of initial entity node encodings corresponding, respectively, to the first plurality of entity nodes, each initial entity node encoding indicating one or more initial attributes of the entity represented by the respective entity node, a second plurality of initial interaction node encodings corresponding, respectively, to the second plurality of interaction nodes, each initial interaction node encoding indicating one or more initial attributes of the interaction represented by the respective interaction node, and a plurality of initial edge encodings corresponding, respectively, to the plurality of directed edges, each initial edge encoding indicating one or more initial attributes of the respective directed edge, and wherein the class data include one or more class encodings representing one or more respective classes of the biological system, each class encoding indicating one or more attributes of the respective class of the biological system; and obtaining sample data comprising a plurality of records derived from a respective plurality of samples of the biological system, each record indicating presence, absence, and/or expression levels of one or more of the entities in the respective sample of the biological system; dividing the sample data into a training set and a validation set; providing the biological system data as input to a machine learning model to initialize the machine learning model; training the model to model the biological system based on the training set of the sample data; and validating the trained model using the validation set of the sample data.

According to another aspect of the present disclosure, a biological system modeling method is provided. The modeling method includes obtaining input sample data comprising a record derived from a first sample of a biological system, the record indicating (i) presence, absence, and/or expression levels of one or more entities in the first sample of the biological system, and (ii) one or more first classes to which the first sample of the biological system belongs; providing the input sample data as input to a machine learning model trained to model the biological system, wherein the machine learning model has been initialized using biological system data and trained using training sample data, the biological system data include architectural data (GL1) and class data, the architectural data represent a bipartite graph representing the biological system, wherein (i) the graph includes a first plurality of entity nodes representing a plurality of entities included in the biological system, a second plurality of interaction nodes representing a plurality of interactions between respective subsets of the plurality of entities, and a plurality of directed edges connecting a plurality of node pairs, each node pair including a respective first node representing an entity of the plurality of entities and a respective second node representing an interaction of the plurality of interactions, (ii) the graph is structured as a closed-loop control system, and (iii) the architectural data (GL1) include an architectural encoding including a first plurality of entity node encodings corresponding, respectively, to the first plurality of entity nodes, each entity node encoding indicating one or more attributes of the entity represented by the respective entity node, a second plurality of interaction node encodings corresponding, respectively, to the second plurality of interaction nodes, each interaction node encoding indicating one or more attributes of the interaction represented by the respective interaction node, and a plurality of edge encodings corresponding, respectively, to the plurality of directed edges, each edge encoding indicating one or more attributes of the respective directed edge, the class data include one or more class encodings representing one or more respective classes of the biological system, each class encoding indicating one or more attributes of the respective class of the biological system, and the training sample data comprise a plurality of records derived from a respective plurality of second samples of the biological system, each record indicating presence, absence, and/or expression levels of one or more of the plurality of entities in the respective second sample of the biological system; and determining one or more attributes of the first sample of the biological system based on output of the machine learning model.

The above and other preferred features, including various novel details of implementation and combination of events, will now be more particularly described with reference to the accompanying figures and pointed out in the claims. It will be understood that the particular systems and methods described herein are shown by way of illustration only and not as limitations. As will be understood by those skilled in the art, the principles and features described herein may be employed in various and numerous embodiments without departing from the scope of any of the present inventions. As can be appreciated from the foregoing and the following description, each and every feature described herein, and each and every combination of two or more such features, is included within the scope of the present disclosure provided that the features included in such a combination are not mutually inconsistent. In addition, any feature or combination of features may be specifically excluded from any embodiment of any of the present inventions.

The foregoing Summary is intended to assist the reader in understanding the present disclosure, and does not in any way limit the scope of any of the claims.

### BRIEF DESCRIPTION OF THE DRAWINGS

These and other features, aspects, and advantages of the present invention will become better understood with regard to the following description, and accompanying drawings, where:

FIG. 1 shows a schematic biological system simulation pipeline, in accordance with an embodiment.

FIG. 2 is an example block diagram depicting the deployment of a biological system model, in accordance with an embodiment.

FIG. 3 is an example bipartite graph representing a biological system as a closed loop control system, in accordance with an embodiment.

FIG. 4A depicts L1 protein embedding vectors from Prot-T5 plotted in 2 dimensions via dimensionality reduction techniques, in accordance with an embodiment.

FIG. 4B depicts L1 gene embedding vectors from Hyena and DNABERT plotted in 2 dimensions via dimensionality reduction techniques, in accordance with an embodiment

FIG. 5 depicts an example flow diagram for modeling the dynamics of the interaction between a tissue, disease, and drug, in accordance with an embodiment.

FIG. 6 is an example modular general, powerful, scalable (GPS) graph transformer, with examples of positional encoding and structural encoding, in accordance with an embodiment.

FIG. 7 is a flowchart of a process for training a biological system model, in accordance with an embodiment.

FIG. 8 is a flowchart of a process for deploying a biological system model, in accordance with an embodiment.

FIG. 9 depicts an example computing device for implementing the system and methods described in reference to FIGS. 1-8, in accordance with an embodiment.

FIG. 10 depicts a correlation for transcript levels predicted by a biological system model compared to actual of the log2-fold of transcript levels in NSCLC, in accordance with an embodiment.

FIG. 11 depicts correlation coefficients for normalized protein levels in the disease state in NSCLC predicted by the trained model compared to actual protein levels, in accordance with an embodiment.

FIG. 12 depicts receiver operating characteristic (ROC) curves obtained from predicting bound or not-bound drug-target pairs by using a binding model, in accordance with an embodiment.

FIG. 13 depicts correlation coefficients for between actual and predicted transcript levels for six EGFR inhibitors in lung, in accordance with an embodiment.

FIG. 14 depicts predicted protein levels and actual protein levels in lung cancer (NSCLC) tissue using a trained disease model, in accordance with an embodiment.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

It will be appreciated that, for simplicity and clarity of illustration, where considered appropriate, reference numerals may be repeated among the figures to indicate corresponding or analogous elements. In addition, numerous specific details are set forth in order to provide a thorough understanding of the exemplary embodiments described herein. However, it will be understood by those of ordinary skill in the art that the exemplary embodiments described herein may be practiced without these specific details.

### Definitions

Terms used in the claims and specification are defined as set forth below unless otherwise specified.

The phrase “biological system” refers to a biological organization that may span several scales (or levels of organization). Examples of biological systems include, but are not limited to, molecular systems (e.g., molecular signaling cascades), cells, tissues, organs, and/or organ systems. Examples of biological systems also include, without limitation, cells, cellular organelles, macromolecular complexes, and regulatory pathways. In some examples, the phrase “biological system” refers to two or more constituent biological systems interacting with each other (e.g., a cell interacting with a therapeutic agent).

The phrase “particular state” of a biological system refers to a state of the biological system that can be identified by a set of observable or detectable characteristics, such as protein expression levels for certain proteins or upregulation of apoptosis-related genes. In some embodiments, a biological system may be classified into different states from different points of view. In one example, from a disease point of view, a biological system can be classified into a healthy state, disease state, untreated state, or treated state.

The phrase “healthy state” refers to a state of a biological system such as a cell or tissue that is absent of one or more disease-related phenotypes, genotypes, or impairment. For example, if an individual is absent of one or more disease-related phenotypes, genotypes, or impairment, a tissue or a cell obtained from the individual is considered to be in a healthy state. In another example, if a tissue is absent of one or more disease-related phenotypes, genotypes, or impairment, a cell obtained from that tissue is considered to be in a healthy state.

The phrase “disease state” refers to another state of a biological system. In various embodiments, the disease state refers to a presence of a disease-related phenotype or genotype associated with the biological system. For example, if a tissue or organ has a certain diseased phenotype or genotype, a cell in the tissue or organ may be considered in a disease state. In some embodiments, the disease of a cell or another biological system may be indicated based on the presence of cytotoxicity, growth inhibition, and/or apoptosis in the biological system.

The phrase “untreated/treated state” refers to yet another state of a biological system. In various embodiments, a biological system previously in a disease state may be treated with a certain treatment agent or drug. After a certain period of treatment, the biological system may be absent of the disease (e.g., a disease-related phenotype or genotype) and thus is considered as in a treated state. On the other hand, if the biological system is still present with certain disease-related features (e.g., updated or downregulated protein expression and/or disease-related phenotype or genotype). the biological system is considered in an untreated state.

The phrase “therapeutic agent” or “treatment agent” or “drug” refers to a chemical substance, typically of known structure, which, when administered to a living organism or biological system, produces a biological effect. A drug can be a small molecule, a nucleic acid molecule (e.g., vector (e.g., viral vector, non-integrating vector), short interfering RNA (siRNA), microRNA (miRNA), short hairpin RNA (shRNA), antisense oligonucleotide. nuclease, transposon, and aptamer), an antibody or antibody fragment, a peptide, or a protein. In some embodiments, a drug can be a plant or animal extract with an unknown structure.

The phrase “biological system model” refers to a machine learned model configured to predict values of certain observable or detectable attributes of a biological system, to simulate a biological system (e.g., simulate the dynamic behavior of and interactions between components of a biological system), or to otherwise model the biological system. In some examples, a biological system model is configured to predict transcript and/or protein expression levels of a biological system. In some embodiments, the biological system model is configured to predict values of attributes of a biological system with a disease state perturbed by a treatment agent. In some embodiments, the biological system model is configured to predict a state of a biological system. In some embodiments, the biological system model is configured to predict the effects of a treatment agent on a biological system. In some embodiments, the biological system model is configured to predict one or more cellular behaviors associated with a biological system under one or more possible conditions.

The term “omics data” refers to data from one or more modalities such as genomics, transcriptomics, epigenetics, metabolomics, and/or proteomics, indicating the presence, absence, expression level, and/or activation of genes, metabolites, proteins, and/or transcripts within a biological system. The term “multi-omics data” refers to data from two or more modalities such as genomics, transcriptomics, metabolomics, epigenetics, and/or proteomics. Multi-omics data generally enables a more comprehensive understanding of molecular changes contributing to normal development, cellular response, and disease. Using integrative omics technologies, a model can better connect genotype to phenotype and fuel the discovery of novel drug targets and biomarkers. In some embodiments, omics data comprises data from one or more cell lines or from a patient-derived sample.

### Motivation for and Applications of Some Embodiments

While the governing equations of most biological systems remain unknown, there have been significant advances in understanding how and which components of biological systems are coupled. The coupling between these components can be represented in the form of a graph (e.g., bond graph). In addition, significant amounts of omics data have been collected, for example, in the form of transcript-, gene-, and prote-omics data. Such data effectively stand in as observables of biological systems of drug, disease, and tissue. What is needed is a mechanical formalism by which the different components are coupled and a model of how the components interact to regulate the behavior of a biological system and produce the observables.

The inventors have appreciated that one mechanical formalism that describes (or models) the coupling of the components of biological system is an optimal control loop. A biological modeling system incorporating this formalism may resemble a system of coupled ordinary differential equations where the specific symbolic dependencies remain unknown. The inventors have further appreciated that data-driven representations of these dependencies can be obtained using machine learning techniques, thereby avoiding the difficulty of deriving specific symbolic forms for these dependencies. In some examples, such data-driven representations are learned using neural networks powered by modern deep-learning approaches. For example, the coupled components of a biological system may be represented as a directed graph, and the coupling between the nodes of the graph may be established via message-passing neural networks. This approach enables a gray-box methodology for understanding and modeling the behavior of biological systems (e.g., cells) in the presence of a disease and a drug where there is transparency and interpretability that is provided ab-initio due to the optimal control and graph formalisms, while still allowing the flexibility and scale provided by the use of neural networks.

First-principles modeling of biological system dynamics (e.g., cellular dynamics) is a complicated and challenging task, as it involves integrating knowledge from various fields such as biology, physics, and mathematics. Drugs failing in clinical trials due to inefficacy or toxicity do so because the cellular and preclinical assays only capture limited aspects of the dynamics of the disease, and there is a lack of translatability from assay to human. For example, assays are frequently carried out in immortalized cell lines that do not reflect the signaling cascades and genetic alterations present in the disease state. These cell lines are used because they are easy to maintain in a laboratory setting. Furthermore, these assays provide limited information about a compound and its impact on cellular dynamics. The results of these assays may tell a scientist whether a drug has activated or inhibited a particular mechanism, but do not provide information about the individual proteins and molecules that were perturbed. More recently, the availability of multi-omics data has engendered the ability to quantify more aspects of the state of a biological system.

In the present disclosure, a biological system modeling approach (e.g., cellular simulation approach) is developed, whereby a biological system (e.g., a human, a tissue, a cell, or a set of cells) is modeled from the molecular level to the organismal level. providing molecular context at each level for the state of the biological system or the effect of a drug on the system. At a high level, this approach can be described by the following steps: 1) Define the biological system, e.g., identify the biological system of interest and define the scope of the model. For example, the model may focus on a particular cell, a particular state of a cell, a particular cellular process, such as the cell cycle, and so on. 2) Identify the relevant biological entities (e.g., genes, transcripts, proteins, and so on) and the interactions therebetween. 3) Develop mathematical models of the interactions between and among the biological entities. For example, based on the identified interactions, a mathematical model may be developed that describes the system's behavior. The development of the model may involve using differential equations or other mathematical models (e.g., machine learned models) to simulate the system's dynamics. 4) Parameterize the model. The model's parameters may be determined (e.g., machine learned) based on experimental data, such as protein levels, reaction rates, or diffusion constants, as described in detail below. 5) Validate the model. For example, the model may be validated by comparing its predictions with experimental observations. This validation step may involve testing the model's predictions against new experiments or comparing the model's predictions with previously published data. 6) Use the model to make predictions. For example, once the model is validated, it may be used to make predictions about the system's behavior under different conditions.

Disclosed herein are some embodiments of specific methods and systems for implementing the above described biological system modeling approach (e.g., cellular simulation approach). For example, disclosed herein are some embodiments of methods and systems for modeling biological system behavior (e.g., cellular behavior) at scale using graph neural networks. The associated system may be referred to herein as a “biological system simulation pipeline”). In some embodiments, the biological system simulation pipeline may include one or more machine learned models for predicting unknown entity levels (e.g., transcript and/or protein levels) within the biological system based on known omics data (e.g., transcriptomics and/or proteomics data) for the healthy and/or diseased state of the biological system (e.g., cell). In some embodiments, the biological system simulation pipeline may facilitate identification of the underlying mechanisms of health activity and/or diseased activity within the biological system. In some embodiments, the biological system simulation pipeline may facilitate identification and testing of drug candidates for certain diseases by simulating a diseased biological system's interaction with the drug candidate.

In various embodiments, the biological system simulation pipeline may represent the biological system using a graph (e.g., bond graph). The graph may include nodes representing entities within the biological system and nodes representing certain interactions (e.g., biological, chemical, physical, or electrical interactions) between and among those entities. In one example, the graph disclosed herein is a bipartite graph that includes a first set of nodes representing entities within a biological system and a second set of nodes representing entity interactions within the biological system. For example, to simulate the cellular behavior of a cell using the disclosed simulation pipeline, genes, transcripts, and proteins of the cell may be represented by entity nodes in the graph, while the gene transcription events and messenger RNA translation events that govern entity interactions may be represented by entity interaction nodes in the graph.

In some embodiments, to simulate the behavior of a biological system that includes the sheer number of complex entity interactions inside the graph, one or more machine learning models (e.g., graph neural networks) are developed. During the model-training process, these models can learn data-driven representations of these interactions from experimentally collected data. For example, in some embodiments of the simulation pipeline, one or more graph neural networks may be built upon and operate on the suitably defined bipartite graph to capture the dynamics of the interactions among the biological system's entities (represented by the graph's nodes). Each of the as-built graph neural networks may have one or more message passing layers such that the graph nodes iteratively update their representations by exchanging information with their neighbors.

After initializing the machine learning model (e.g., graph neural network(s)) based on the bipartite graph, experimental data may be used to further train the model to transform the biological system's initial encodings, such that the updated encodings represent system dynamics learned during the training process. For example, the initialized model can be trained using the experimentally collected data, such that nodes exchange information with their neighbors, thereby progressively transforming the initial system architecture (based on the training set of the experimentally collected data) to obtain an updated architecture indicating one or more updated attributes of the entity nodes and one or more updated attributes interaction nodes. The updated attributes of the entity nodes and one or more updated attributes of interaction nodes may more accurately represent the dynamics of the biological system.

In various embodiments, using sample data collected from instances of the biological system in different states, different models corresponding to different states of the biological system can be generated through the training process. For example, a trained model may model (1) a healthy biological system (e.g., cell) if trained on samples from healthy instances of the biological system (e.g., healthy cells), (2) a diseased biological system if trained on samples from diseased instances of the biological system, or (3) a treated biological system if trained on samples from instances of the biological system treated with a therapeutic agent. In some embodiments, for each disease and/or for each tissue type, there may be a model trained for such purposes. Accordingly, in applications, different models may be trained depending on the goals of the models to be developed. It should be noted that, in some embodiments, a unified model can be trained under various conditions (e.g., trained using samples of healthy or diseased biological systems from different tissues, biological systems afflicted with different diseases, and/or biological systems treated with different drug treatments). Such a unified model can be applied to model or simulate behavior of a biological system under various conditions.

In some embodiments, the model of a biological system may provide outputs indicating one or more predicted (or inferred) expression levels of one or more of the plurality of entities of the biological system. In some embodiments, the model of a biological system may be used to determine one or more mechanisms of action of the biological system, and/or to determine one or more pharmacokinetic and/or pharmacodynamic properties of one or more of the plurality of entities of the biological system, as described below.

Embodiments of the methods and systems disclosed herein offer certain benefits and advantages. For example, some embodiments provide predicted measurements of expression levels of certain observables (e.g., proteins, transcripts, metabolites, and the like) within a biological system that would otherwise be obtained only using expensive and time-consuming wet lab measurement tools. In addition, some embodiments facilitate and reduce the expense of drug discovery, drug testing, diagnosis of disease, personalized machine, more comprehensive assessment of the side effects of drugs, and more comprehensive assessment of the effects of using multiple drugs simultaneously.

Incorporating expert knowledge into the structure of the machine-learning model via the arrangement and interconnection of nodes representing entities and interactions in a graph, and incorporating the optimal control loop formalism into the topology of the graph, enhances the efficiency of the machine-learning techniques used to train the model. Thus, some embodiments can train a biological system model to reach a specified level of accuracy or performance far more efficiently (e.g., using less time and/or fewer computational resources) than is possible with modeling techniques that do not incorporate the graph and/or the optimal control loop formalism.

It should be noted that the features and benefits described herein are not all-inclusive, and many additional features and benefits will be apparent to one of ordinary skill in the art in view the following descriptions of specific embodiments.

### Methods and Systems for Modeling Biological Systems, and Applications Thereof

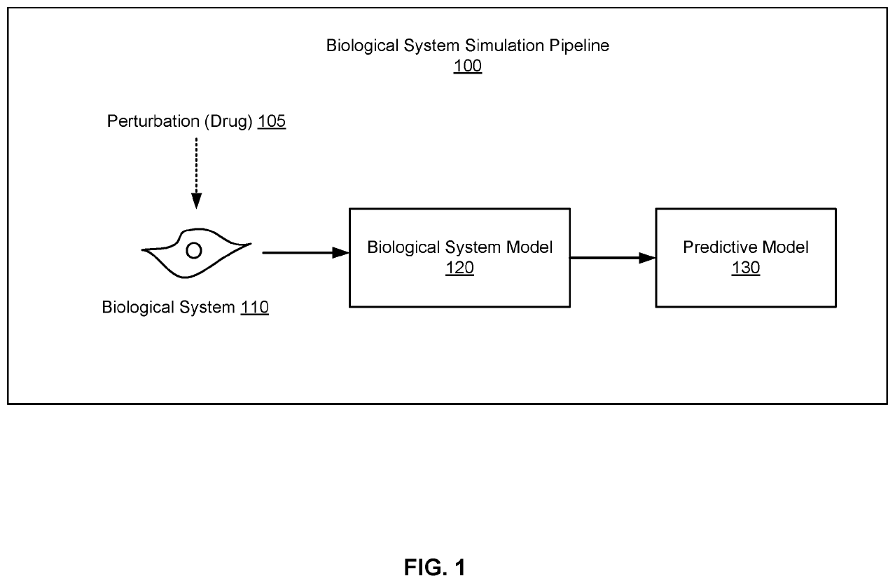

FIG. 1 depicts an example biological system simulation pipeline 100, in accordance with an embodiment. Generally, the simulation pipeline 100 includes a biological system 110 (e.g., a cell) that is to be analyzed.

In various embodiments, biological system 110 can be a cell extracted from a tissue or organ that exhibits tissue-or organ-specific features, including but not limited to specific phenotypes. In various embodiments, the cell may be in a healthy state or diseased state. For example, the tissue or organ from which the cell is sampled may be in good health or may be in a diseased state. In various embodiments, the cell can be in an untreated or treated state. For example, the cell may have been in a diseased state, and after applying a perturbation such as a drug 105 to the cell, the cell's state may have changed from the diseased state to a treated state, which may be the same as or different from the cell's healthy or diseased state. In various embodiments, the cell can be sampled from a person who shows single nucleotide polymorphisms (SNPs) in certain genes. In some embodiments, these SNPs may affect the efficacy of a drug 105 in disease treatment.

Although not shown, the disclosed simulation pipeline 100 may include one or more devices for obtaining (e.g., measuring) omics data (e.g., multi-omics data) from one or more samples of the biological system. Such samples may be obtained, for example, from the same cell line, from the same organism's tissue, or from the same type of tissue in other organisms. The one or more devices for obtaining (e.g., measuring) the omics data may include a first device for obtaining transcriptomics data, a second device for obtaining proteomics data, a third device for obtaining epigenetics data, and so on. In some embodiments, the disclosed simulation pipeline 100 may not have a device for measuring the omics data. Instead, the simulation pipeline 100 may obtain the data from other sources. e.g., from a third-party service provider, from other institutions, from databases or literature, or from online sources. For example (and without limitation), the omics data may be obtained from the genotype-tissue expression (GTEx) project, ENCODE, GEO, TCGA, CPTAC, DepMap, Expression Atlas, Human Cell Atlas, Human Protein Atlas, PRIDE, Allen Brain Map, gNOMAD, dbGaP, cBioPortal, recount2, UK Biobank, CCLE, ARCHS4, and/or CREEDS.

In various embodiments, the simulation pipeline 100 further includes a biological system model 120 configured to model the biological system 110. For example, the biological system model 120 may use one or more graph neural networks to model the dynamics of the biological system 110 by taking into consideration local entity features and dynamic entity interaction features, as well as global tissue and/or disease features. For example, when provided with disease state information and tissue information, a trained biological system model 120 may model the behavior of a biological system (e.g., cell) from that tissue and having that disease. For example, the model 120 may simulate one or more protein expressions for that biological system (e.g., cell). Additionally or alternatively, based on certain known features identified from the biological system 110, the model 120 may infer certain unknown of the biological system 110. For example, certain “unseen” (e.g., undetected/unreported) protein expressions may be inferred based on certain “seen” (e.g., detected/reported) protein expressions. (Due to detection limits or errors, the expression levels of some proteins are generally not detected in a proteomics analysis. Similarly, transcriptomics does not necessarily provide information on every transcript.) By using some embodiments of the model 120, a more comprehensive understanding of the behavior of a biological system can be achieved

In various embodiments, the simulation pipeline 100 further includes a predictive model 130 configured to make one or more predictions based on the outputs of the biological system model 120. The predictive model 130 may be a machine-learned model, a mathematical model, or any other suitable type of model. For example, the predictive model 130 may predict whether the behavior of a sample of the biological system 110 shows some changes when compared to other samples of the biological system 110 (e.g., control group). As another example, the predictive model 130 may compare the expression levels of entities of the biological system 110 in a diseased state with the expression levels of entities of the biological system in a healthy state. Based on the comparison, the predictive model 130 may infer certain mechanisms underlying certain diseases. For example, if the differences identified through the comparison are related to a specific pathway in a cell, the predictive model 130 may infer that the disease is related to that specific pathway.

In another example, the predictive model 130 may compare the expression levels of entities of a biological system in a diseased state before and after a drug 105 treatment. (The drug treatment may be carried out physically, or may be simulated using the biological system model 120.) Based on the comparison, the predictive model 130 may determine whether drug 105 has the potential to effectively treat the disease. For example, if the expression levels of entities of the biological system after the drug treatment are comparable to the expression levels of the same entities in a healthy instance of the biological system (e.g., within ranges associated with healthy cells), the predictive model 130 may determine that drug 105 has the potential to treat the disease.

It should be noted that while the predictive model 130 is illustrated as a separate unit different from the biological system model 120, in some embodiments, the predictive model 130 and the biological system model 130 may be integrated into the same unit (e.g., into a same neural network or set of neural networks). For example, a graph neural network may include certain layers for biological system modeling and certain layers for prediction based on entity expression levels. The outputs from the biological system modeling layers (which may be graph embedding layers) may be used for prediction by the prediction layers.

### Biological System Model

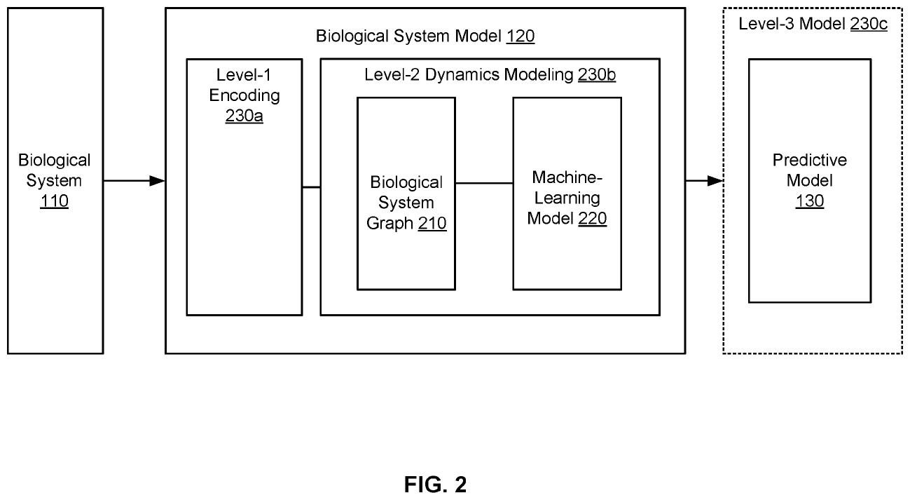

Referring to FIG. 2, in the biological system model 120, the components of a biological system 110 and the relationships therebetween are represented as a directed heterogenous bipartite graph, where physical components (also referred to as “entities”) are represented by one set of nodes and the interactions between the components are represented by the second set of nodes. Compared to other graphs covering limited aspects of a biological system, some embodiments of biological system graph 210 do not artificially delineate between data modalities. Instead, described herein is an extended graph framework for enhanced coverage of biological system dynamics (e.g., cellular function). For example, some embodiments of the extended graph framework 210 disclosed herein substantially increase coverage over the ontological space of components.

As also illustrated, in connection with the graph 210 (e.g., bipartite bond graph), the biological system model 120 includes machine-learning model 220 built upon the graph 210, which itself is associated with a set of encoders 230a for encoding or embedding the features of entities or the relationships between the entities included in the graph 210. For example, given that attending over atomic space for simulation at the organism scale is Impractical because of compute limitations, it is desirable to have a set of level-1 encoders 230a that can capture these useful features in a lower-dimensional space. In modeling or simulation, projecting biological components into vector space is desirable, where each component modality may be historically associated with a set of tasks, which capture the important properties of the component within a vector representation. For example, a key task for proteins is physical conformation and structure, small molecule tasks often center on quantum mechanical property prediction, and transcript and gene primary sequence tasks, being similar to natural language processing (NLP) tasks, generally center on reconstruction. In each case, these components and their associated properties define and govern the higher-order relationships modeled by the graph. In some embodiments, the encoders described herein are capable of capturing the structural, domain-specific, and ontological features of the corresponding components. Additional tasks serve to regularize the latent vector space for each domain, such that these features are captured.

In some embodiments, proteins and small molecules can be structured as graphs, genes and transcripts can be structured as sequences, and reactions and their associated kinetics values can be defined by their neighbors within the graph. This formulation, therefore, uses a limited number of encoding architectures to cover the complete set of modalities. Once encoded into the vector space, these components form the input layer of the graph which contains topological information. Information at the level of component structure and function is able to propagate up to the higher order graph, the structure and topology of which informs predictions for higher order tasks. Accordingly, the level-2 model 230b disclosed herein may propagate the information throughout the graph, evolve the state, and even perform regression on the state to predict transcript or protein levels at their respective nodes.

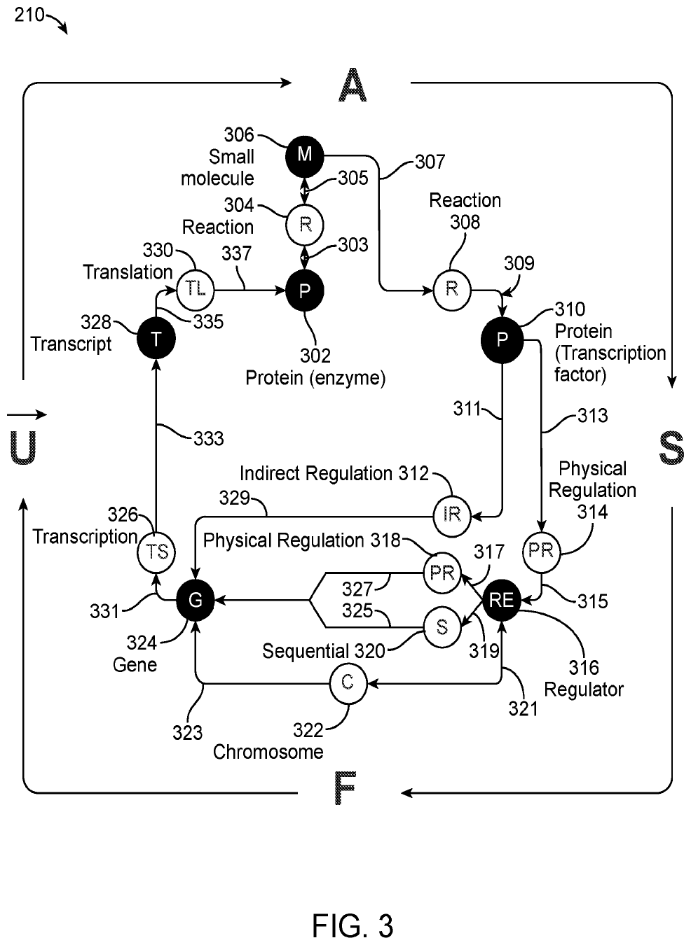

In some embodiments, the graph disclosed herein may optionally include a level-3 model 230c that predicts the pharmacokinetic properties of a treated state if a drug is applied to a disease in a treated state. For example, under certain circumstances, the level-3 model 230c may be used to predict pharmacokinetic properties such as absorption, distribution, metabolism, excretion, and toxicity (ADMET), based on the outputs of the level-2 model, e.g., based on the predicted expression levels of certain proteins in a biological system. The specific functions of different components in the biological system model 120 are further described in detail with reference to FIGS. 3-6.

In some embodiments, the “as-built” (e.g., initialized) graph may be provided as input to or built into a machine-learning model 220. For example, the machine learning model 220 may include one or more graph neural networks, which may include nodes corresponding to entity nodes in the graph. The graph neural networks may include certain layers that can update their representations by exchanging information with their neighbors. In this way, the graph neural networks may cooperatively function as a machine learning model 220 that includes certain functions to exchange information with neighbor nodes, such as the update function. aggregation function, etc. The machine learning model 220 may be thus a dynamic model that can be trained to better capture the dynamic behavior of a biological system. The functions and components of the biological system model 120 are further detail in detail hereinafter.

### Biological System Graph

In some embodiments, the biological system graph 210 is a bond graph. A bond graph is a graphical representation of a dynamic system (e.g., a biological system). Bond graphs allow the conversion of the dynamic system into a state-space representation. In this way, a bond graph is similar to a block diagram or signal-flow graph. The arcs (edges) in bond graphs can represent unidirectional or bi-directional interaction (e.g., exchange of physical energy, flow of information, etc.). A bond graph can incorporate multiple domains seamlessly.

Bond graphs can be used to represent complex relationships and interactions between entities in a wide range of domains, including biology. In particular, bond graphs representing biological (e.g., cellular) systems provide a powerful tool for analyzing the complex relationships between “actors” (e.g., biological entities) within the biological system (e.g., cell). While existing knowledge graphs can provide useful tools for modeling biological systems, there are some unresolved challenges in applying these knowledge graphs to real-world scenarios. For instance, databases of biological information tend to reflect the experimental techniques used to generate the information. As a result, each type of biological component is typically characterized in databases that are siloed, that is, each type of biological component is stored in isolation with respect to other types of data components. For example, transcriptomics data are decoupled from proteomics data. However, given the complex and interconnected nature of these regulatory networks, it can be challenging to study these dynamics in an isolated manner. In the embodiments disclosed herein, the biological system graph 210 is configured to model the biological system as a whole. with biological entities of the biological system represented by nodes, interactions between the biological entities represented by nodes, and the nodes interconnected by edges. An example biological system graph configured for a cellular system (e.g., a specific cell from a tissue or organ) is described in detail below.

In an example biological system graph for a cellular system or a cell is a heterogeneous graph containing about 1.2 million nodes. The graph is configured beyond just representing entities (e.g., genes, transcripts, or proteins) within the cellular system as nodes. Instead, the graph may capture complex interactions between different entities within the biological system. For example, the graph may include interaction nodes representing interactions involving two or more entities within the biological system. Accordingly, among the ˜1.2 million nodes in the example graph, a first subset of nodes represent the entities within the biological system, and a second subset of nodes represent the interactions between the entities. That is, the graph may have a bipartite graph structure, with two distinct sets of nodes representing entities and interactions, respectively.

FIG. 3 illustrates an example biological system graph 210 for a cellular system, according to one embodiment. In the illustrated graph, the two distinct types of nodes are represented by different colors. The grey nodes represent the entity nodes and the white nodes represent the interaction nodes in the graph. In the illustrated graph, only representative nodes or node types are illustrated, where these representative nodes or node types are categorized based on the structure and/or function of the entities or interactions they represent. For example, in the graph 210, a set of proteins (e.g., the proteins working as enzymes) are shown as a single node “P” (302) in the graph 210. In a real cell, the set of proteins represented by the node “P” (302) (e.g., protein-type enzymes) may be large, and thus the protein node “P” (302) in the graph 210 can actually represent a large number of protein nodes (e.g., protein enzyme nodes). The specific features of each node in the graph 210 are further described in detail below.

The protein node “P” (302) in the graph 210 represents a set of one or more proteins (e.g., a set of proteins functioning as enzymes) inside a cellular system (e.g., a human cell). In one example, a protein is a macromolecule consisting of long chains of amino acids (AAs). The AA sequence determines the complex 3D protein folding structure, which in turn determines the protein's function. Proteins are the chief actor within a cellular system, performing many biological functions, such as enzymes functioning as biological catalysts. The fundamental representation of a protein enzyme can be an amino acid sequence. As will be described later, the fundamental representation of the entity represented by each node can be used to encode features of the entity represented by the node for processing by a machine-learning model (e.g., one or more graph neural networks).

The gene node “G” (324) in the graph 210 represents a set of one or more genes (e.g., all genes) inside a cellular system. In one example, a gene is a region of DNA that encodes a function. The role of a gene in this context is to be transcribed to produce a functional RNA. There are two types of molecular genes: protein-coding genes and noncoding genes. Genes are made up of DNA, more specifically, four types of genetic bases including adenine (A), cytosine (C), guanine (G), and thymine (T). In humans, genes vary in size from a few hundred DNA bases to more than 2 million bases. The fundamental representation of a gene can be a DNA sequence.

The transcript node “T” (328) in the graph 210 represents a set of one or more transcripts transcribed from genes in the cellular system. In one example, a transcript is a single-stranded RNA product synthesized by transcription of DNA, and processed to yield various mature RNA products such as mRNAs, tRNAs, and rRNAs. The transcripts designated to be mRNAs are modified in preparation for translation wherein proteins are produced. As described below, the transcription of genes is regulated by different regulation factors. The fundamental representation of a transcript can be an RNA sequence.

The node “M” (306) in the graph 210 represents a set of one or more small molecules (e.g., metabolites) inside a cellular system. In one example, a small molecule is a low molecular weight compound, typically involved in a biological process as a substrate or product. The small molecules in the cellular system may include sugars, lipids, amino acids, fatty acids, phenolic compounds, alkaloids, etc. The fundamental representation of a small molecule or metabolite can be a simplified molecular-input line-entry system (SMILES) sequence. SMILES is a specification in the form of a line notation for describing the structure of chemical species using short ASCII strings. SMILES strings can be imported by most molecule editors for conversion back into two-dimensional drawings or three-dimensional models of the molecules.

Another protein node “P” (310) in the graph 210 represents a set of one or more proteins (e.g., proteins functioning as transcription factors) inside a cellular system. In one example, a transcription factor (TF) (or sequence-specific DNA-binding factor) is a protein that controls the rate of transcription of genetic information from DNA to messenger RNA, by binding to a specific DNA sequence. Similar to proteins functioning as enzymes, the proteins functioning as transcription factors also consist of chains of amino acids, and the fundamental representation of a transcription factor can be an amino acid sequence.

The regulator node “RE” (316) in the graph 210 represents a set of one or more regulatory regions in the genome that regulate other genes. In one example, a regulator gene (sometimes referred to herein as a “regulator” or “regulatory gene”) is a gene involved in controlling the expression of one or more other genes, such as enhancers, promoters, repressors, etc. A regulator gene may encode a protein, or it may work at the level of RNA, as in the case of genes encoding microRNAs. An example of a regulator gene is a gene that codes for a repressor protein that inhibits the activity of an operator (a gene that binds repressor proteins thus inhibiting the translation of RNA to protein via RNA polymerase). Compared to regulator genes represented by the regulator node “RE”, the genes represented by the gene node “G” may represent non-regulatory genes. Similar to the genes represented by the gene node “G”, the fundamental representation of a regulatory gene can be a DNA sequence.

It should be noted that, while six types of entity nodes are illustrated in graph 210, a biological system (e.g., cellular system) may include entity node types not illustrated in graph 210, according to some embodiments. For example, besides functioning as enzymes and transcription factors, certain proteins in a cellular system may function as muscle or body tissue building units (i.e., structural proteins), as some types of hormones that regulate the actions of other proteins or other molecules, as transport units for maintaining fluid and/or electrolyte balance and acid-base (pH) balance and for transporting nutrients, as antibodies in an immune system, etc. These various types of proteins may be represented by other entity nodes not illustrated in the graph 210.

In another example, a biological system (e.g., cellular system) may include a set of one or more complexes, which can be represented by nodes (“complex nodes”) not shown in the graph 210. A biomolecular complex is made of multiple biomolecules interacting non-covalently. Complexes may be multiprotein complexes, RNA-protein complexes, DNA-protein complexes, and protein-lipid complexes. Alternatively, a biomolecular complex may be represented by a neighborhood of nodes in the graph 210. In the latter case, the fundamental representation of a biomolecular complex can be a neighborhood (e.g., a set of nodes representing the biomolecules that make up the biomolecular complex), or can include the fundamental representations of the biomolecules that make up the biomolecular complex.

In yet another example, a biological system (e.g., cellular system) may include a set of one or more physical entities that represent modified proteins. Thus, the graph may include physical entity nodes that each represent a protein that has been modified (e.g., phosphorylated), altering the function of the protein. The fundamental representation of a modified protein is a modified AA sequence.

Referring back to FIG. 3, besides the entity nodes, the graph 210 further includes a set of interaction nodes, which are depicted in FIG. 3 using white discs. Each representative interaction node represents a type of interaction between the entities within a biological system (e.g., cellular system).

The reaction node “R” (304) in graph 210 represents a set of one or more biochemical reactions between entities within a cellular system. In one example, a biochemical reaction is the transformation of one molecule (e.g., an amino acid) to a different molecule (e.g., a peptide) inside a cellular system. Biochemical reactions are mediated by enzymes, which are biological catalysts that can alter the rate and specificity of chemical reactions inside cells. The fundamental representation of a biochemical reaction can be a differential equation (DE), one or more structural encodings, and/or one or more positional encodings. The reaction node “R” (308) in graph 210 also represents biochemical reactions between entities within a biological system. Non-limiting examples of biochemical reactions include enzymatic reactions, transcription factor reactions, reactions between proteins and metabolites, etc.

The transcription node “TS” (326) in the graph 210 represents a set of one or more processes of transcribing genomic DNA into RNAs. In general, transcription is a process of transcribing a segment of DNA into RNA molecules, some of which can further encode proteins. In one example, a gene is transcribed into a transcript, which may be represented using the notation gene→transcription→transcript. The fundamental representation of a transcription process can be an ordinary differential equation (ODE), one or more structural encodings, and/or one or more positional encodings.

The translation node “TL” (330) in the graph 210 represents a set of one or more processes of translating messenger RNAs (mRNA) into proteins. In general, mRNA is decoded in a ribosome, outside the nucleus, to produce a specific amino acid chain. In one example, a transcript (e.g., mRNA) is translated into a protein, which may be represented using the notation transcript→translation→protein. The fundamental representation of a translation process can be an ODE, one or more structural encodings, and/or one or more positional encodings.

The physical regulation node “PR” (314 or 318) in the graph 210 represents a set of one or more processes by which gene expression is regulated (e.g., gene transcription). For example, a physical regulation node 314 or 318 may represent regulation of gene expression from cis-or trans-regulatory elements (e.g., enhancers, promoters and/or some proteins that can regulate gene expression). The fundamental representation of a physical regulation process can be an ODE, one or more structural encodings, and/or one or more positional encodings.

The indirect regulation node “IR” (312) in graph 210 represents one or more processes for indirect regulation of gene expression. For example, the rate of transcription of genetic information from DNA to messenger RNA may be controlled by the binding of a transcription factor (TF) (or sequence-specific DNA-binding factor) (a protein) to a specific DNA sequence. The fundamental representation of an indirect regulation process can be an ODE, one or more structural encodings, and/or one or more positional encodings.

Sequential connection node “S” (320) in graph 210 represents a set of one or more bindings or connections between regulators (such as an enhancer) in one region of the genome and another region of the genome. The fundamental representation of a sequential connection can be one or more structural encodings and/or one or more positional encodings.

Chromosome connection node “C” (322) in graph 210 represents a set of one or more chromosomal interactions or connections that regulate gene expression. For example, gene expression can be controlled by regulators that can be located far away along the chromosome or in some cases even on other chromosomes. That is, complex genome-wide networks of chromosomal interactions can regulate gene expression. The fundamental representation of a chromosome-based regulation process can be an ODE, one or more structural encodings, and/or one or more positional encodings.

Though not shown, the graph 210 can include interaction nodes representing certain other interactions between entities. In one example, pathway nodes may represent interactions between different entities, such as different molecules. In general, a pathway is a series of events/interactions that regulate the state of a biological system (e.g., cell). A pathway typically has a stimulus or signal that leads to downstream events that alter the location, state, function, and/or concentration of biological system components (e.g., cellular components). The fundamental representation of a pathway can be coupled DEs, one or more structural encodings, and/or one or more positional encodings. In another example, degradation nodes may represent interactions between proteins or RNAs in their original forms and degraded forms. For example, the lifetime of mRNA molecules is usually short, while proteins may have different lifetimes. The fundamental representation of degradation can be half-life, one or more structural encodings, and/or one or more positional encodings.

In some embodiments, the graph 210 disclosed herein is constructed as a closed-loop control system (e.g., optimal control loop), where each node in the graph may have a different role in the closed-loop control system. In control theory, the state-space of a system, e.g., the speed of a car in cruise control, is modified by a control, e.g., the acceleration and deceleration mechanisms, to optimize an objective function, e.g., to maintain a certain speed. In a closed-loop control system, the control is generated via a feedback process, e.g., whether to accelerate or decelerate, that leverages sensory data, e.g., the speedometer. Similarly, for healthy biological systems (e.g., cells), the objective of the system (e.g., cell) can be to maintain the state-space of the system in homeostasis. To reflect this objective, the entity nodes and interaction nodes in the graph 210 can be constructed as an optimal control loop, where different nodes play different roles in the control system. In one example, the different nodes in the graph 210 can be categorized as parts of the control system's state-space, control framework, feedback framework, and/or sensor framework. For example, as shown in FIG. 3, the state-space of the control system can include the gene, transcript, and protein nodes (e.g., protein node 302), the transcription nodes and translation nodes can form the control vector \vec{u} of the control system, the actuator (A) of the control system can include the reaction nodes (304, 308) and metabolite nodes. The remaining nodes in graph 210 (including, in some examples, protein nodes 310) can be categorized as either sensors “S” or feedback “F” in the control system.

The graph 210 shown in FIG. 3 is a high level illustration of different entity nodes and interaction nodes, which also shows a high level connectivity in a closed-loop block diagram. In general, the specific categorization of each node in the control system is based on the function it performs and that function's relationship to the cell's objective of maintaining homeostasis. Furthermore, one of ordinary skill in the art will appreciate that different biological systems can be represented by different graphs 210.

In some embodiments, to simulate how a cell maintains homeostasis like a control system, it may be desirable to understand the equations that govern the entity nodes and interaction nodes. However, considering the sheer number of dynamic quantities to tracked results in a system of O(100,00) coupled differential algebraic equations (DAEs). Numerical integration of these quantities is intractable, even if numerical stability constraints are met. For this reason, in the present disclosure, instead of using differential algebraic equations to model the microscale dynamics, a machine learning model (e.g., one or more graph neural networks) is used, which learns data-driven representations of the system dynamics experimentally collected data.

### Modeling Techniques

In some embodiments, to learn the models that govern the dynamics of the biological system represented by the graph 210, a machine-learning model is used. In some embodiments, the machine learning model includes one or more graph neural networks (GNNs). The graph neural network is a class of artificial neural networks for processing data that can be represented as graphs. GNNs are well-suited for operating on graph structures and capturing relationships between different entities. Thus, GNNs are suitable for modeling relationships among entities represented by different nodes in a biological system graph 210. In some embodiments, GNNs use pairwise message passing, such that graph nodes iteratively update their representations by exchanging information with their neighbors. Such message passing allows information to propagate throughout the graph. By leveraging the power of GNNs in combination with the biological system graph disclosed herein, the complex interactions and dependencies between entities in a biological system can be learned through the process of training the GNNs (e.g., using experimentally obtained data). The trained GNNs can be then used to make predictions about the biological system represented by the biological system graph under different conditions. In addition, the trained model of a biological system provides a deeper understanding of the underlying mechanisms driving overall behavior of the biological system. In one example, by modeling the effect of drug candidate on a cellular system and predicting the resulting state of the cellular system, some embodiments facilitate identification of potential drug targets for the treatment of various diseases.

In some embodiments, the modeling of a biological system can be conceptually divided into two levels, level-1 (or “L1”) and level-2 (or “L2”). In some embodiments, the modeling of a biological system can be conceptually divided into three levels, L1, L2, and level-3 (or “L3”). Briefly, in level-1, local information about entities represented in the graph 210 is encoded (e.g., embedded into node feature vectors) via various encoders. In level-2, a machine learning model (e.g., one or more GNNs) propagates information through the graph 210, evolves the state of the system, and perform regressions on the state to predict levels of observables (e.g., proteins, transcripts, etc.) represented by respective nodes. In level-3, additional models may generate additional predictions based on the outputs of the level-2 model. For example, in the case of modeling interactions between a drug candidate and a biological system, the pharmacokinetic properties of the biological system in the treated state can be predicted at level-3. In the following sections, the details of each level in the modeling approach are further described.

### Level-1: Input Encoding

As described herein, the biological system graph 210 is a bipartite heterogeneous graph that includes two types of nodes (i.e., entity nodes and interaction nodes). In some embodiments, the biological system graph can be expressed as follows:

$$G = ( X , E , τ , R ) \qquad ( 1 )$$

where X=U∪V is a set of nodes (U representing the set of entity nodes and V representing the set of interaction nodes in the graph 210), E is a set of edges (e.g., directed edges) connecting elements of U and V, R is a set of node-types and τ is a node-type mapping, τ: x^{(r)}→r∈R∀x∈X. Edges represents relationships between the nodes. In some embodiments, the edges can be directed, where an edge has a source node and a destination node. For a directed edge, the information flows from the source node to the destination node. For example, through the set of directed edges in the example of graph 210 shown in FIG. 3, the information processed by the model flows in the directions shown by the arrows in the graph.

In some embodiments, each entity represented by an entity node in the graph 210 is fundamentally represented by a sequence, whether they be nucleotide sequences for DNA/RNA, amino-acid sequences for proteins, or SMILES strings for molecules. However, for many types of machine learning models (e.g., GNNs) to ingest this node-level information, the sequences are first encoded as features (e.g., embedded into fixed-length feature vectors). In level-1 modeling, to encode the local information about entities represented by entity nodes in the graph 210, a set of encoders (ϕr) are applied to the entity nodes, where each encoder corresponds to a sequence type. In some embodiments, the encoders embed each node's local information into a fixed-length feature vector. In one example, the encoders applied to the entity nodes may be expressed as follow:

$$ϕ r : x ( r ) \to {\vec{x}}^{( r )} ∈ RFr \qquad ( 2 )$$

where Fr is the feature space dimension for node-type r. This encoding process can be repeated for all node-types such that the entity node attributes are transformed as

$${L}_{1} : U \to {U}_{L 1} = {⋃}_{r ∈ R} \{ ϕ r ( x ) \vert x ∈ X ∧ τ : x \to r \} \qquad ( 3 )$$

In some embodiments, the encodings (e.g., embeddings) generated by the encoders allow the level-2 model to leverage the local sequence information at each entity node in addition to the graph structure to make predictions about the system's behavior. This modeling approach is especially valuable in the realm of biological systems, where an entity's sequence can significantly impact its functionality and interactions with other entities. With this in mind, it is helpful for the sequence encoders to understand the language of a given sequence type so the decoders can extract the relevant information about the structure and function of the underlying entities. To this end, transformer-based encoder models may be used. These encoder models may be pre-trained on large corpora of the relevant sequences.

In some embodiments, masked language modeling (MLM) is used in the transformer-based encoder models. The use of MLM may be particularly helpful in scenarios in which the relevant sequence information for certain entities is missing (e.g., unknown or unavailable). Masked language modeling is a type of deep learning in which a model learns from large amounts of unlabeled sequences by predicting the sequences that are randomly masked in the input. For example, if the sequence information is missing for an entity in a biological system, masked language modeling can be applied to generate a probable sequence for the entity.

In some embodiments, the transformer-based encoder models are applied to sequences of the entities, to extract the encodings (e.g., embedding vectors) from the latent state vectors (or context vectors) of the transformer. The transformer-based models (or simply “transformers”) generally use an encoder-decoder architecture. The encoder extracts features from an input sequence, and the decoder uses the features to produce an output (translation). In the embodiments disclosed herein, a transformer-based encoder model may include an encoder configured for constructing a fixed-length latent vector (or context vector) for an input sequence. The transformer-based model may also include a decoder configured to use the latent vector to (re)construct a feature encoding (e.g., embedding vector) as the output. In some embodiments, both the input sequence and output feature encoding (e.g., embedding vector) can be of variable lengths. In the following, the exemplary gene, transcript, and protein encodings are further described.

In some embodiments, a transformer-based model (e.g., “Prot-T5”) is employed to encode protein sequences. Prot-T5 is a protein language model pre-trained on a large database of 2.5 billion sequences, fine-tuned on Uniref50) (45 million sequences), and developed using the T5 (text-to-text transfer transformer) architecture. The Prot-T5 architecture is suitable for generating encodings (e.g., embedding vectors) of protein sequences due to its encoder-decoder design, as well as its excellent performance on many downstream tasks without the need for fine-tuning. In some embodiments, other transformer-based models can be also used in generating the encodings (e.g., embedding vectors) for the proteins.

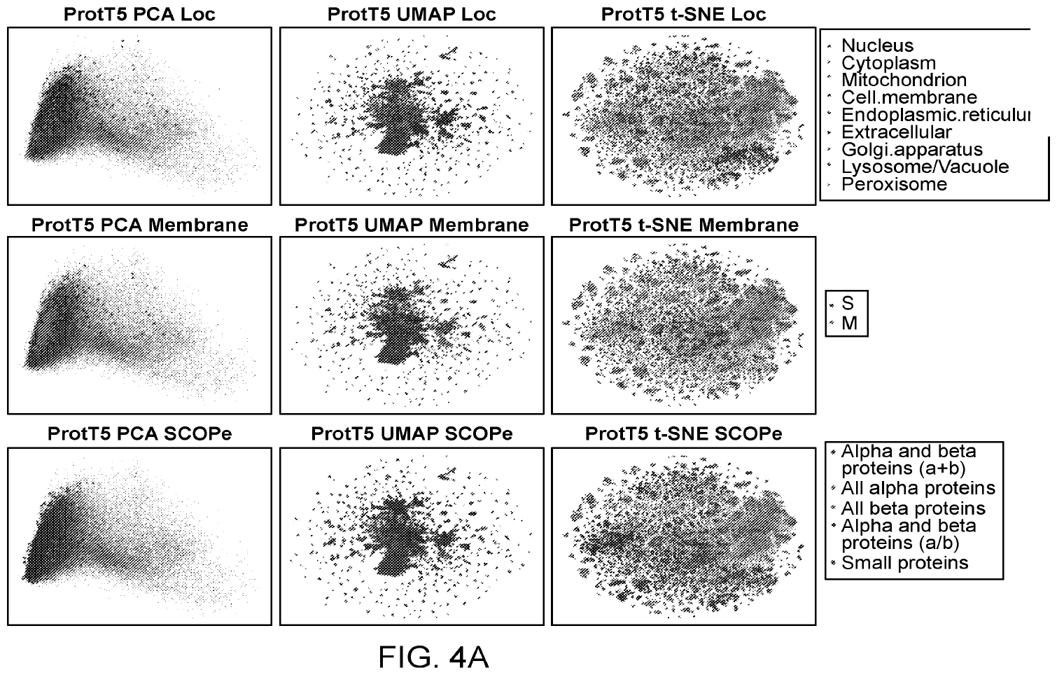

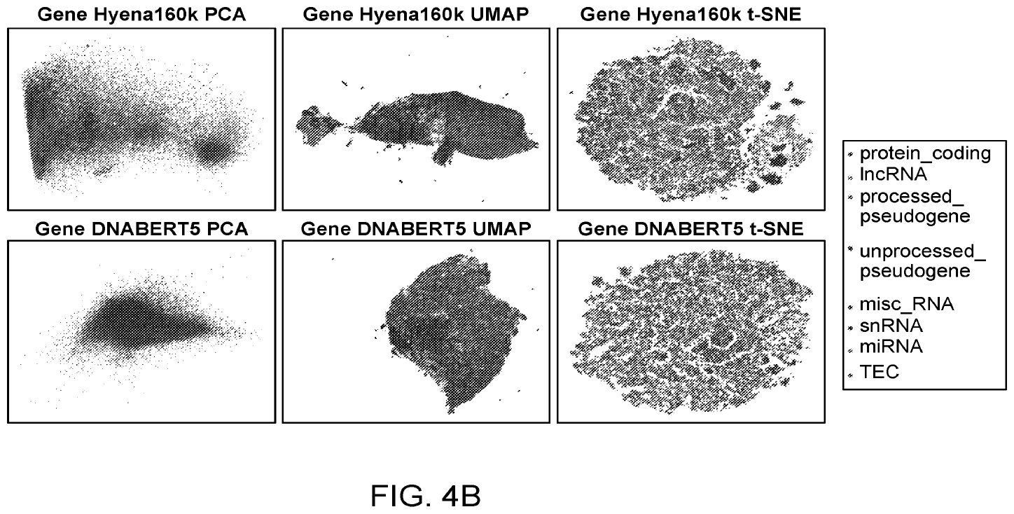

FIG. 4A illustrates L1 protein embedding vectors from Prot-T5 plotted in two dimensions via dimensionality reduction techniques, such as principal component analysis (PCA), uniform manifold approximation and projection (UMAP), and t-distributed stochastic neighbor embedding (t-SNE). Feature extraction methods such as PCA, t-SNE, and UMAP reduce dimensionality in datasets in such a manner that the low dimensional space is a good representation of the original (or high) dimensional space. In FIG. 4A, proteins are colored according to their cellular location, membrane solubility, and structural classification of proteins—extended (SCOPe) category.

In some embodiments, a transformer-based model (e.g., DNA bidirectional encoder representation, or “DNABERT”) is used to encode both gene and transcript information. DNABERT is configured to capture the global and transferrable understanding of the genomic DNA sequences based on up-and down-stream nucleotide contexts. When properly trained, DNABERT may simultaneously achieve excellent performance on the prediction of promoters, splice sites, and transcription factor binding sites, after easy fine-tuning using small task-specific labeled data. Further, DNABERT enables direct visualization of nucleotide-level importance and semantic relationships within input sequences for better interpretability and accurate identification of conserved sequence motifs and functional genetic variant candidates. Finally, pre-trained DNABERT with a human genome can even be readily applied to other organisms and many other sequence analysis tasks with exceptional performance.

In some embodiments, in an existing DNABERT, a 512-token context window is employed, which may not be suitable for encoding DNA/RNA sequences having different lengths. To address this concern, in some embodiments, encodings (e.g., embeddings) are generated for DNA/RNA chunks, instead of the full DNA/RNA sequence, within the window, and the chunks are then averaged to produce the full sequence. It is to be noted, in some embodiments, the encodings (e.g., embeddings) generated in this manner may suffer from context loss between the different chunks. To address this issue, much larger context windows (e.g., Hyena filters) may be used instead. A Hyena operator uses a parametrized convolution to help deep learning models understand relationships between different pieces of information (e.g., different DNA/RNA chunks within a full sequence).

FIG. 4B illustrates L1 gene embedding vectors from Hyena and DNABERT plotted in two dimensions via dimensionality reduction techniques (PCA, UMAP, and t-SNE). In FIG. 4B, genes are colored according to their Ensembl biotype category, which includes protein coding, IncRNA, unprocessed pseudogene, processed pseudogene, misc_RNA, snRNA, miRNA, and TEC.

In some embodiments, transformer-based models may be used to encode sequence information for other entities represented by entity nodes, such as small molecules (e.g., metabolites). In some embodiments, small molecule encodings are obtained from publicly available libraries, such as molfeat.

In some embodiments, attributes of the graph's interaction nodes may also be encoded (e.g., using structural and/or positional encodings), as described below.

In some embodiments, the L1 encoding process may involve obtaining encodings of classifications assigned to the biological system. Examples of biological system classifications may include tissue type, disease type (if the system is diseased), or treatment agent (if the treatment of the biological system with a therapeutic agent is being modeled). In some embodiments, the encodings of the system's classifications may be global feature embeddings, as described below.

In some embodiments, the L1 encoding process may involve obtaining encodings of binding scores (e.g., for each protein in the system, scores indicating the probability that each one of a set of therapeutic agents binds the protein). In some embodiments, the encodings of the system's binding scores may be node-specific feature embeddings, as described below.

In some embodiments, the tissue and disease information can be embedded as global feature vectors, denoted as \vec{u}ₜᵢₛₛᵤₑ and \vec{u}_{disease}, respectively. This approach facilitates parameterizing the healthy and diseased states across many tissues and diseases as H(\vec{u}ₜᵢₛₛᵤₑ) and D(\vec{ }\vec{u}ₜᵢₛₛᵤₑ, \vec{u}_{disease}), where “H” represents health states and “D” represents diseased states.

In some embodiments, a language model (LM) may be used to generate embedding spaces for the tissue and disease information. Pre-trained language models (e.g., BERT, GPTs) have shown remarkable performance on many natural language processing tasks, such as text classification and question answering. By performing self-supervised learning, such as masked language modeling, LMs can learn to encode various knowledge from text corpora and provide informative representations of tissue and disease for downstream tasks.

In some embodiments, a BioLinkBERT model may be used for space embedding for the tissue and disease information. BioLinkBERT is a transformer encoder (BERT-like) model pre-trained on a large corpus of documents. An improvement of BERT, BioLinkBERT newly captures document links such as hyperlinks and citation links to include knowledge that spans across multiple documents. Specifically, it is pre-trained by feeding linked biomedical literature into the same language model context, rather than just non-linked documents. For example, PubMed (which is a free full-text archive of biomedical and life sciences journal literature at the U.S. National Institutes of Health's National Library of Medicine) abstracts can be used as inputs to extract the embedding vectors, which are averaged across the literature for each tissue/disease. The resulting embedding space is informed by biomedical knowledge, which allows encoding the physiological and pathological context of the system, thereby improving the generalization and predictive capabilities of the model.

In some embodiments, the ontology for tissue and disease may be input into the tissue or disease encoder for generating ontological feature vectors for a specific tissue or disease. The tissue or disease encoder can be any of the pre-trained language models described above, for example, the BioLinkBERT model, and the generated ontological feature vectors may carry the disease-and tissue-specific features, which can be used to parameterize the healthy and diseased states across many tissues and diseases as H(\vec{u}ₜᵢₛₛᵤₑ) and D(\vec{ }\vec{u}ₜᵢₛₛᵤₑ, \vec{u}_{disease}), respectively.

To assist with modeling of the effects on the biological system of therapeutic agents, drug candidates, or other perturbations, some embodiments may generate encoding of therapeutic agents, drug candidates, or other perturbations. In the case of the therapeutic agents and drug candidates, the agent/drug candidate can be any biological entity that acts on the biological system (e.g., cellular system) by binding to certain entities (e.g., proteins) inside the biological system. Some examples of agents/drug candidates may include, without limitation, small molecules, nucleic acid molecules (e.g., a vector (e.g., viral vector), short interfering RNA (siRNA), microRNA (miRNA), short hairpin RNA (shRNA), antisense oligonucleotide, nuclease, transposon, and/or aptamer), an antibody or antibody fragment, and/or a peptide. In the following paragraphs, the encoding (e.g., embedding) for a small molecule is described for exemplary purposes, but the described encoding and modeling steps can be applied to any therapeutic agent, drug candidate, or other biological perturbation.

Most small molecule drugs are designed to bind to specific proteins and modulate their activity, either enhancing or inhibiting their function, ultimately leading to a desired therapeutic effect. Therefore, protein-drug binding affinities can encode the impact of a drug on a biological system to a first order.

In some embodiments, a binding model may be used to generate binding scores for protein nodes (or other entity nodes such as a gene nodes, metabolite nodes, or regulator nodes) for a given set of drug compounds, to encode the effects of the drugs on the biological system at the entity (node) level. In one example, this binding information can be denoted as:

$$B = \{ \vec{p} ( x ) \vert x ∈ X ∧ τ : x \to protein \} \qquad ( 4 )$$

where \vec{p}(x) is a vector of binding probabilities for a protein x across the space of drug compounds. This information can then be propagated by the machine learning model (e.g., one or more GNNs) to inform the model about potential downstream interactions and predict corresponding changes in observables (e.g., transcriptomic observables, proteomic observables, etc.). This approach facilitates a deeper understanding of treatment dynamics in vivo including the potential off-target effects.

### Level-2: System Dynamics

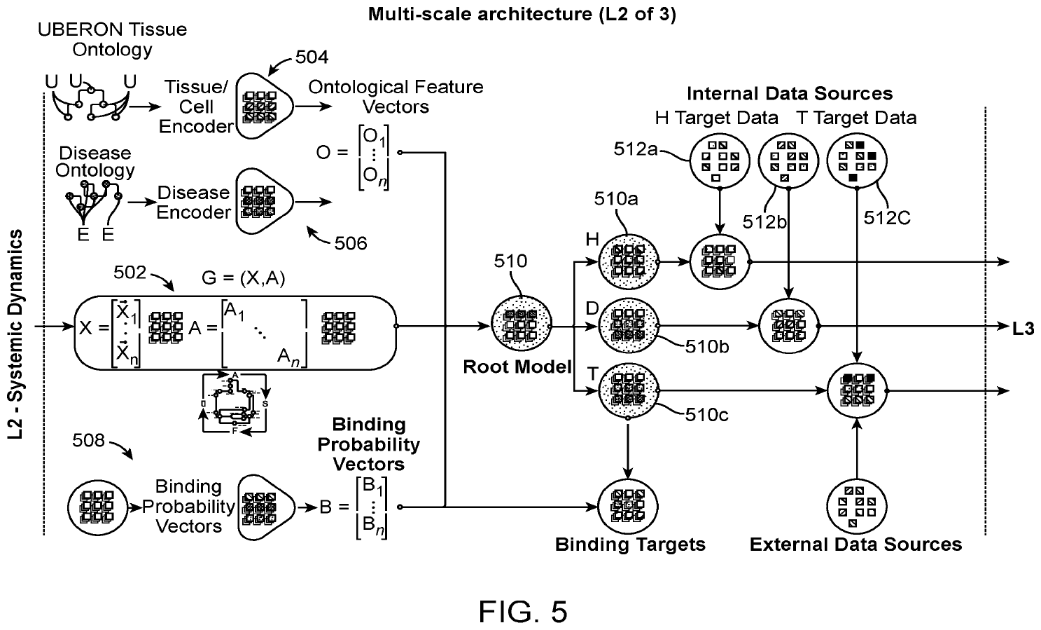

FIG. 5 illustrates the level-2 modeling of a biological system's dynamics using the level-1 encodings. As described above, through the level-1 processing, the biological system model 120 is enriched with the encoded node features (e.g., fixed-length vectors), which encode local information that can be denoted as:

$${G}_{L 1} = ( {U}_{L 1} ⋃ V , E , τ , R ) \qquad ( 5 )$$

The graph encoding G_{L1} (502) may be referred to herein as the “as-built” graph or the “initialized” graph. In some embodiments, the level-1 encodings include global feature encodings (e.g., vectors), which may encode one or more classifications associated with the biological system (e.g., the physiological/pathological context in which to view the system). For example, the level-1 encodings may include a tissue encoding (504) and/or a disease encoding (506). In some embodiments, the level-1 encodings include a set of binding encodings (e.g., binding vectors) (508) which encode possible drug interactions with proteins in the graph.

In the level-2 portion of the biological system model 120, the L1 encodings are provided as inputs to a machine learning model (e.g., one or more graph neural networks). During the process of training the machine learning model, the propagation of the information represented by the L1 encodings through is governed by the graph 210. After being trained, the level-2 model is configured to predict one or more observables (e.g., expression levels of one or more transcriptomic observables, proteomic observables, transcripts-per-million up-regulation or down-regulation of proteins, etc.).

FIG. 5 illustrates an example architecture for level-2 modeling of a biological system, according to some embodiments. In some embodiments, during a process of training the model, the level-1 encodings are propagated through the graph 210 and transformed at each step using message passing neural networks (MPNNs), yielding a trained model 510. Depending on the class encodings (504, 506, 508) and training data (512a, 512b, 512c) used during the training process, the trained model may be a healthy state model (or simply “healthy model”) (510a) configured to model the biological system in a health state, a diseased state model (or simply “disease model”) (510b) configured to model the biological system in one or more diseased states, a treated state model (or simply “treated model”) (510c) configured to model treatment of the biological system with one or more drugs, or a unified model capable of modeling the biological system in a healthy state, modeling the biological system in one or more diseased states, and/or modeling treatment of the biological system with one or more drugs.

For example, a healthy state model incorporates information (504) regarding the tissue type (504) of the biological system, diseased state models additionally incorporate information (506) related to the diseases present in the biological system, and the treated state models further incorporate information (508) regarding the drug used to treat the biological system. For example, as shown in FIG. 5. ontological tissue and disease feature encodings may be incorporated into the diseased state model. For the treated model, beyond the ontological tissue and disease feature encodings, binding probability encodings and/or binding targets identified based on the binding probability encodings may be further incorporated into the treated state model.

In some embodiments, healthy, diseased, and treated models can be respectively expressed as:

$$H ( {\vec{u}}_{t i s s u e} ) = P r i m a r yModel ( {G}_{L 1} , {\vec{u}}_{t i s s u e} ) \qquad ( 6 )$$

$$D ( {\vec{u}}_{t i s s u e} , {\vec{u}}_{d i s e a s e} ) = PrimaryModel ( GL 1 , {\vec{u}}_{t i s s u e} ⌢ {\vec{u}}_{disease} ) \qquad ( 7 )$$

where [symbol] represents the concatenation of two vectors. In the above Equations (6) and (7), the operations inside PrimaryModel(G, \vec{u}) for a given input G=(U∪V, E, τ, R), which is a subgraph of G_{L1}, and a condition vector \vec{u} may operate as follows. First, entity node features may be updated using input multi-layer perceptrons (MLPs) for each node type as:

$${U}^{′} = \{ M L P r ( \vec{x} ) \vert \vec{x} ∈ UL 1 ∧ τ : \vec{x} \to r ∈ R \} \qquad ( 8 )$$

Second, the global condition vector, \vec{u}, may be concatenated with the current node features as

$$X = \{ \vec{x} ⌢ \tilde{u} \vert \vec{x} ∈ {U}^{′} ⋃ V \} \qquad ( 9 )$$

In the case of the treated state model, the conditioning may take place at the protein node-level rather than globally, hence the conditioning function is:

$$x ( drugi ) = \{ \vec{x} j ⌢ ( \vec{p} j ) i \vert \vec{p} j ∈ B \vert \vec{x} j ∈ {U}^{′} ∧ τ : \vec{x} j \to protein \} ⋃ \{ \vec{x} ∈ {U}^{′} ∧ τ : \vec{x} \to protein \} ⋃ V \qquad ( 10 )$$

where i is a drug index, and j is a protein index.

In some embodiments, the training process for the healthy, diseased, treated, and/or unified models described above is supervised using node-level regression or classification tasks against publicly available and/or privately provided omics data (e.g., transcript-and prote-omics data. etc.). This methodology provides a gray-box approach that enables mechanistic interpretability while still maintaining the flexibility provided by data-driven approaches that leverage machine learning models (e.g., neural networks). To achieve this objective, the biological system model disclosed herein may include a final output layer that is task dependent. The purpose of the final output layer may be to provide (e.g., decode) the output used for supervising the model in the training process. In some embodiments, the final output layer is configured as follows:

$${K}_{i} ( {\vec{x}}^{j} ) = {K}_{i} ( H ( {\vec{x}}^{j} , θ H ) , θ {K}_{i} ) \qquad ( 11 )$$

where Kᵢ is the i^{th} task. In some embodiments, the various instantiations of the equation (11) are supervised during the training of the model. This formulation allows the training system to supervise a node type differently per dataset. In other words, this approach permits different tasks to condition the same node type.

Referring again to FIG. 5, in some embodiments, node-level features are not encoded for the interaction nodes that do not represent physical entities. In some embodiments, positional and/or structural encodings are provided for the interaction nodes of the graph 210, the entity nodes of the graph 210, and/or the edges of the graph 210. Some reasons for providing positional and/or structural encodings are described below.

In general, in many GNNs, the local structure completely determines the node representation. This approach results in a catastrophic failure case where two nodes with the identical local structure are interpreted as being the same by the GNNs, while the entities they represent may perform completely different functions in the biological system. While this failure can be alleviated via deeper, multi-layer, multi-hop message passing networks, those networks present other problems that have to do with propagating information over many hops, often termed vanishing gradients, or the over-squashing phenomenon.

Structural and positional encodings can encode unique characteristics of each node and/or edge. In the most basic case, when all nodes have the same initial features or no features at all, applying positional and structural features helps to distinguish nodes in a graph, assign them with diverse features, and provide at least some sense of graph structure.

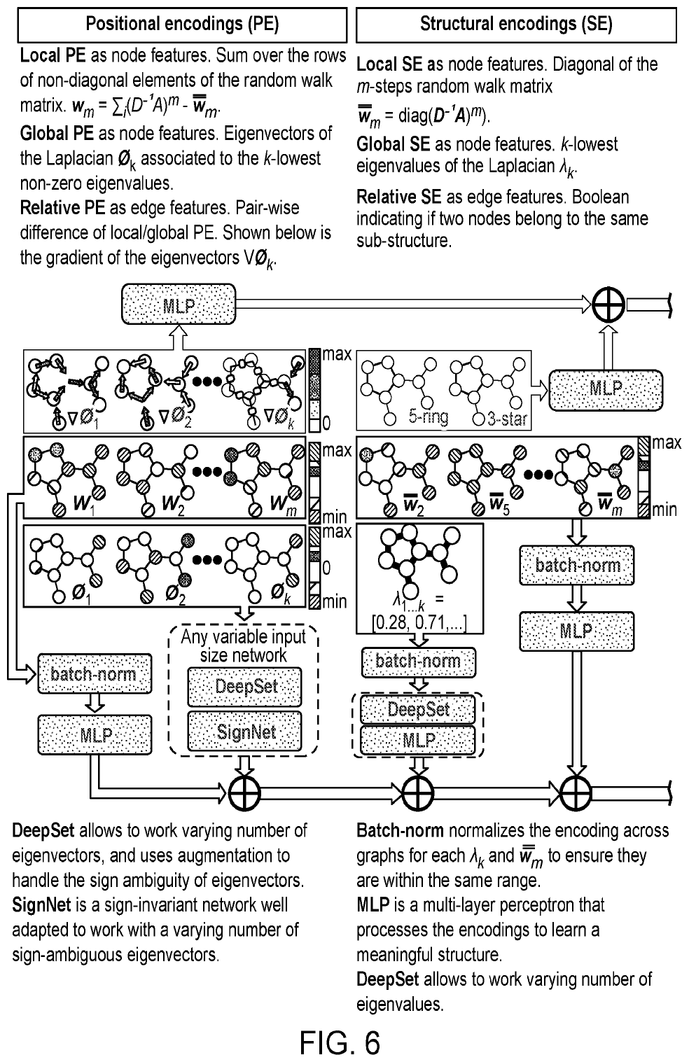

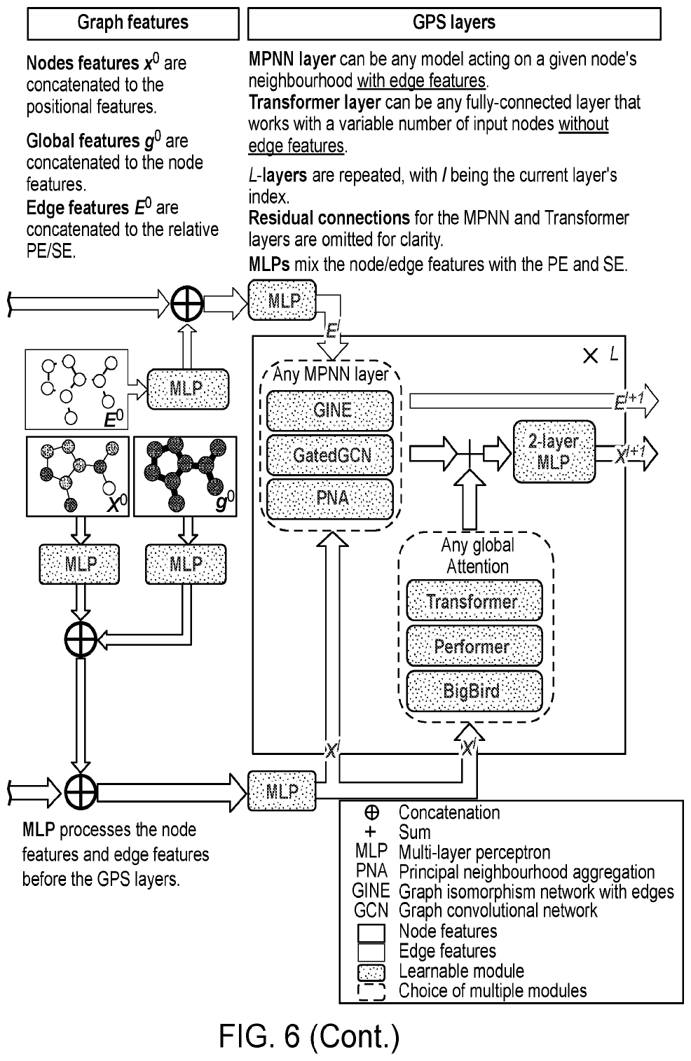

Positional encodings (PE) and structural encodings (SE) that inform each node of its relative position in the entire graph structure can help alleviate the inherent degeneracy in 1-hop networks. It is also important to note that positional encodings are important enablers of the transformer architecture. Because attention operates globally on a sequence where order matters, encoding the relative position of each token in a sequence before inputting it to an attention layer(s) is helpful. As discussed elsewhere herein, some embodiments of the graph 210 involve long range interactions where the distance between annotated nodes can span tens of hops. Computing a positional encoding that uses all nodes and edges of the graph 210 can be computationally expensive. While random walk positional encoding (RWPE) has been traditionally computed with the Laplacian matrix of a given graph, this approach is infeasible for many biological system graphs due to the number of nodes and edges. To solve this problem, RWPE can be approximated using averaging of the landing probabilities from explicit random walks, according to some embodiments. Some embodiments of PEs and SEs for biological system graphs are further described with reference to FIG. 6.

FIG. 6 illustrates an example modular graph transformer with examples of positional encodings and structural encodings. Positional encodings are meant to provide an idea of the position in space of a given node within the graph. Hence, when two nodes are close to each other within a graph or subgraph, their PE should also be close. A common approach of PE is to compute the pair-wise distance between each pair of nodes or their eigenvectors, but this is not compatible with linear transformers as it involves materializing the full attention matrix. Instead, the PEs are expected to be features of the nodes or real edges of the graph, thus a better fitting solution is to use the eigenvectors of the graph Laplacian or their gradient.

Structural encodings are meant to provide an encoding (e.g., embedding) of the structure of graphs or subgraphs to help increase the expressivity and the generalizability of graph neural networks. Hence, when two nodes share similar subgraphs, or when two graphs are similar, their SEs should also be close. Simple approaches of SE are to identify pre-defined patterns in the graphs as one-hot encodings, but they require expert knowledge of graphs. Instead, using the diagonal of the m-steps random-walk matrix encodes richer information into each node, such as for odd m it can indicate if a node is a part of an m-long cycle. Structural encodings can also be used to define the global graph structure, for instance using the eigenvalues of the Laplacian, or as relative edge features to identify if nodes are contained within the same clusters.

As illustrated in FIG. 6, the PEs and SEs can be organized into three categories: local, global, and relative, to facilitate the integration within the pipeline. Local PE as node features allows a node to know its position and role within a local cluster of nodes. Within a cluster, the closer two nodes are to each other, the closer their local PEs will be. Global PE as node features allows a node to know its global position within the graph. For example, the closer two nodes are, the closer their global PE will be. Relative PE as edge features allows two nodes to understand their distances or directional relationships. Edge embedding is correlated to the distance given by any global or local PE, such as the distance between two words.

Local SEs as node features allows nodes to understand what substructures they are part of. Given an SE of radius m, the more similar the m-hop subgraphs around two nodes are, the closer their local SE will be. Global SE as graph features (or node features) provides the network (or nodes) information about the global structure of the graph. The more similar the two graphs are, the closer their global SE will be. Relative SE as edge features allows two nodes to understand how much their structures differ. Edge embedding is correlated to the difference between any local SE.

In some embodiments, the graph-level features are used to update the node-level features. Along with the structural and positional encodings, the graph-level features and node-level features can be passed into a message passing layer and a global attention layer, e.g., a transformer layer as shown in the GPS layer in FIG. 6. The specific steps undertaken may be as follows: First, node features can be updated via an initial heterogeneous message passing layer, (HGNN), as:

$${X}^{′} = H G N N ( X , E , τ , R ) \qquad ( 12 )$$

Second, node features can be updated recursively by n homogeneous hidden GNN layers as:

$$X ( n ) = G N N ( X ( n - 1 ) , E ) \qquad ( 13 )$$

Third, homogeneous node features can be mapped back to the heterogeneous graph structure and a final heterogeneous layer can be applied as:

$$Y = H G N N ( X ( n ) , E , τ , R ) \qquad ( 14 )$$

Fourth, entity node features can be transformed by output MLPs to values representing omic observables (e.g., transcriptomic observables, proteomic observables, etc.) (either single regression outputs or multi-classifier outputs) as:

$${Y}^{′} = \{ M L P r ( \vec{y} ) \vert \vec{y} ∈ Y ∧ τ : \vec{y} \to r ∈ R \} \qquad ( 15 )$$

### Model Training and Deployment

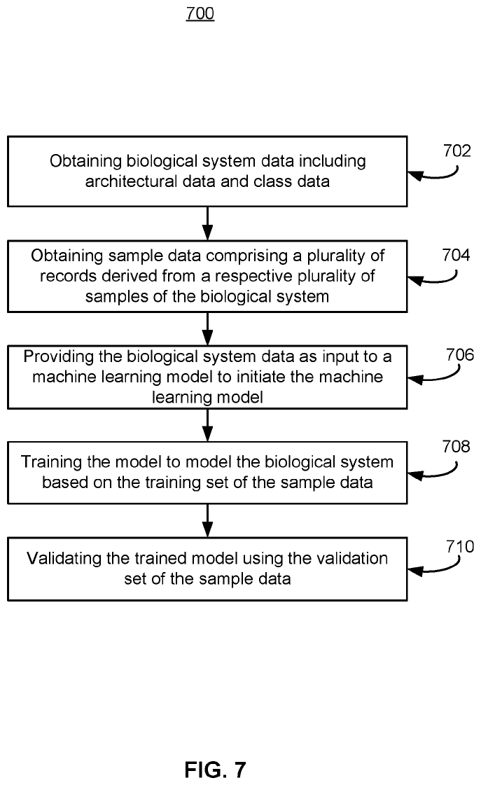

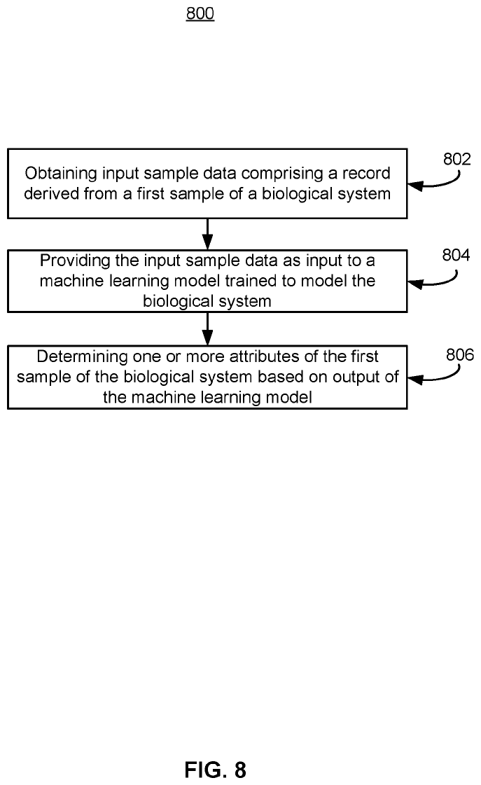

FIG. 7 is a flowchart of a process 700 for training a machine learning model (may be also referred to as a “biological system simulation model” or simply “biological system model”), according to some embodiments. In some embodiments, the biological system model may refer to the biological system model 120 shown in FIGS. 1-2. In some embodiments, the biological system model may refer to both the biological system model 120 and the predictive model 130 in FIGS. 1-2. Furthermore, FIG. 8 is a flowchart of a process for deploying a biological system model trained according to the training process 700, in accordance with an embodiment.

Referring first to FIG. 7, a training process 700 includes step 702 of obtaining biological system data including architectural data (G_{L1}) and class data. The architectural data may represent a bipartite graph representing a biological system. The bipartite graph may include a first plurality of entity nodes representing a plurality of entities included in the biological system, a second plurality of interaction nodes representing a plurality of interactions between respective subsets of the entities, and a plurality of directed edges connecting a plurality of node pairs. Each node pair may include a respective first node representing an entity of the plurality of entities and a respective second node representing an interaction of the plurality of interactions. The bipartite graph may be structured as a closed-loop control system (e.g., optimal control loop). The architectural data (G_{L1}) may include an initial architectural encoding (e.g., a combination of various vectors as shown in FIG. 5 or embeddings or encodings as described earlier in FIGS. 1-6) including a first plurality of initial entity node encodings corresponding, respectively, to the first plurality of entity nodes. Each initial entity node encoding may indicate one or more initial attributes of the entity represented by the respective entity node. The initial architectural encoding may further include a second plurality of initial interaction node encodings corresponding, respectively, to the second plurality of interaction nodes. Each initial interaction node encoding may indicate one or more initial attributes of the interaction represented by the respective interaction node. The initial architectural encoding may further include a plurality of initial edge encodings corresponding, respectively, to the plurality of directed edges. Each initial edge encoding may indicate one or more initial attributes of the respective directed edge.

The class data may include one or more class encodings representing one or more respective classes of the biological system. Each class encoding may indicate one or more attributes of the respective class of the biological system.

The training process 700 may include step 704 of obtaining sample data comprising a plurality of records derived from a respective plurality of samples of the biological system. Each record may indicate presence, absence, and/or expression levels of one or more of the entities in the respective sample of the biological system. In one example, the plurality of records include omics data obtained from the respective plurality of samples of the biological system. The omics data may identify and indicate expression levels of respective subsets of the entities in the plurality of samples of the biological system. For example, the omics data may be single omics data that include data for one type of entity, such as gene, transcript, or protein. Additionally or alternatively, the omics data may be multi-omics data that include data for different types of entities. For example, the omics data may include proteomics data, transcriptomics data, genomics data, metabolomics data, etc. In some embodiments, the obtained omics data can be further divided into a training set and a validation set, which are input into the model at different time points for model training or for model validation, as will be described in detail below.

The training process 700 may include step 706 of providing the biological system data as input to a machine learning model to initiate the machine learning model. That is, the bipartite graph and the initial architectural encoding may be input into the machine learning model (which can be a graph neural network or a set of graph neural networks or other machine learning models that may be built upon the bipartite graph with the initial architectural encoding) to build a machine learning model for modeling the biological system. The machine learning model, when properly built based on the bipartite graph with the initial architectural encoding, may be ready for training.

The training process 700 may include step 708 of training the model to model the biological system based on the training set of the sample data. Here, the training process may include progressively transforming the initial architectural encoding based on the training set of the sample data to obtain an updated architectural encoding.

The training process 700 may include step 710 of validating the model using the validation set of the sample data. For example, the model may be validated based on the accuracy (e.g., correlation coefficient) of predicting unknown protein expressions based on some known protein expressions or transcript expressions and the like. If the accuracy is above a predefined threshold level, the model can be considered valid and can be deployed for applications.

In the following, the various embodiments related to the model training are further described.

In various embodiments, the graph is a biological system graph, as shown in FIG. 3. The plurality of entities include one or more proteins, one or more genes, one or more transcripts, one or more small molecules, one or more biomolecular complexes, and/or one or more regulators, as indicated in the biological system graph shown in FIG. 3. The plurality of interactions include one or more biochemical reactions, one or more transcription events, one or more translation events, one or more physical regulations, one or more indirect regulations, one or more degradations, one or more genomic connections, one or more pathways, and/or one or more chromosomal connections, as also shown in FIG. 3.

In various embodiments, different nodes in the bipartite graph may play different roles in the constructed closed-loop control system. For example, a state-space of the closed-loop control system may be represented by a first group of nodes comprising a fifth subset 324 of the entity nodes representing the one or more genes, a sixth subset 328 of the entity nodes representing the one or more transcripts, and a first subset 302 of the entity nodes representing a first subset of the one or more proteins. One or more actuators of the closed-loop control system may be represented by a second group of nodes comprising a second subset 306 of the entity nodes representing the one or more small molecules, a first subset 304 of the interaction nodes representing a first subset of the one or more biochemical reactions, and a second subset of the interaction nodes 308 representing a second subset of the one or more biochemical reactions. A control vector of the closed-loop control system may be represented by a third group of nodes comprising an eighth subset 326 of the interaction nodes representing the one or more transcription events and a ninth subset 330 of the interaction nodes representing the one or more translation events. One or more sensors and one more feedback units of the closed-loop control system may be represented by a fourth group of nodes comprising a third subset 310 of the entity nodes representing a second subset of the one or more proteins, a fourth subset of the entity nodes representing the one or more regulators 316, a third subset 312 of the interaction nodes representing the one or more indirect regulations, a fourth subset of the interaction nodes 314 representing a first subset of the one or more physical regulations, a fifth subset 318 of the interaction nodes representing a second subset of the one or more physical regulations, a sixth subset 320 of the interaction nodes representing the one or more genomic connections, and a seventh subset 322 of the interaction nodes representing the one or more chromosomal connections.

In various embodiments, the entity nodes are connected to the reaction nodes by directed edges. For example, as shown in FIG. 3, a first subset 302 of the entity nodes representing a first subset of the one or more proteins are connected to a first subset 304 of the interaction nodes representing a first subset of the one or more biochemical reactions by a first subset 303 of the directed edges. A second subset 306 of the entity nodes representing the one or more small molecules are connected to the first subset 304 of the interaction nodes representing the first subset of the one or more biochemical reactions by a second subset 305 of the directed edges. The second subset 306 of the entity nodes representing the one or more small molecules are connected to a second subset of the interaction nodes 308 representing a second subset of the one or more biochemical reactions by a third subset 307 of the directed edges. A third subset 310 of the entity nodes representing a second subset of the one or more proteins are connected to the second subset of the interaction nodes 308 representing the second subset of the one or more biochemical reactions by a fourth subset 309 of the directed edges. The third subset 310 of the entity nodes representing the second subset of the one or more proteins are connected to a third subset 312 of the interaction nodes representing the one or more indirect regulations by a fifth subset 311 of the directed edges. The third subset 310 of the entity nodes representing the second subset of the one or more proteins are connected to a fourth subset of the interaction nodes 314 representing a first subset of the one or more physical regulations by a sixth subset 313 of the directed edges. A fourth subset of the entity nodes representing the one or more regulators 316 are connected to the fourth subset of the interaction nodes 314 representing the first subset of the one or more physical regulations by a seventh subset 315 of the directed edges. The fourth subset 316 of the entity nodes representing the one or more regulators are connected to a fifth subset 318 of the interaction nodes representing a second subset of the one or more physical regulations by an eighth subset 317 of the directed edges. The fourth subset 316 of the entity nodes representing the one or more regulators are connected to a sixth subset 320 of the interaction nodes representing the one or more genomic connections by a ninth subset 319 of the directed edges. The fourth subset 316 of the entity nodes representing the one or more regulators are connected to a seventh subset 322 of the interaction nodes representing the one or more chromosomal connections by a tenth subset 321 of the directed edges. A fifth subset 324 of the entity nodes representing the one or more genes are connected to the seventh subset 322 of the interaction nodes representing the one or more chromosomal connections by an eleventh subset 323 of the directed edges. The fifth subset 324 of the entity nodes representing the one or more genes are connected to the sixth subset 320 of the interaction nodes representing the one or more genomic connections by a twelfth subset 325 of the directed edges. The fifth subset 324 of the entity nodes representing the one or more genes are connected to the fifth subset 318 of the interaction nodes representing the second subset of the one or more physical regulations by a thirteenth subset 327 of the directed edges. The fifth subset 324 of the entity nodes representing the one or more genes are connected to the third subset 312 of the interaction nodes representing the one or more indirect regulations by a fourteenth subset 329 of the directed edges. The fifth subset 324 of the entity nodes representing the one or more genes are connected to an eighth subset 326 of the interaction nodes representing the one or more transcription events by a fifteenth subset 331 of the directed edges. A sixth subset 328 of the entity nodes representing the one or more transcripts are connected to the eighth subset 326 of the interaction nodes representing the one or more transcription events by a sixteenth subset 333 of the directed edges. The sixth subset 328 of the entity nodes representing the one or more transcripts are connected to a ninth subset 330 of the interaction nodes representing the one or more translation events by a seventeenth subset 335 of the directed edges. The first subset 302 of the entity nodes representing the first subset of the one or more proteins are connected to the ninth subset 330 of the interaction nodes representing the one or more translation events by an eighteenth subset 337 of the directed edges.

In various embodiments, the first subset 302 of the entity nodes comprise about 10,000 or more entity nodes representing the first subset of the one or more proteins. In various embodiments, the fifth subset 324 of the entity nodes comprise about 20,000 or more entity nodes representing the one or more genes.

In various embodiments, for each initial entity node encoding in the first plurality of initial entity node encodings, the one or more initial attributes of the respective initial entity node encoding include a first attribute indicating an entity type of the entity represented by the respective entity node. The entity type may refer to a protein, gene, transcript, small molecule, biomolecular complex, modified protein, or regulator. In various embodiments, for each initial entity node encoding in the first plurality of initial entity node encodings, the one or more initial attributes of the respective initial entity node encoding include a second attribute indicating an identity of the entity represented by the respective entity node. In various embodiments, a first subset of the first plurality of initial entity node encodings represent a first subset of the one or more proteins. For each initial entity node encoding in the first subset of the initial entity node encodings, the second attribute indicating the identity of the entity represented by the respective entity node comprises an amino acid sequence or an encoding of the amino acid sequence, and the one or more initial attributes of the respective initial entity node encoding include a third attribute indicating presence, absence, or expression level of the entity represented by the respective entity node.

In various embodiments, the encoding of the amino acid sequence is generated with a PROT-T5 encoder. The encoding of the nucleic acid sequence is generated with a DNABERT encoder or a Hyena filter.

In various embodiments, a second subset of the first plurality of initial entity node encodings represent the one or more metabolites. For each initial entity node encoding in the second subset of the initial entity node encodings, the second attribute indicating the identity of the entity represented by the respective entity node comprises a set of SMILES strings or an encoding of the set of SMILES strings.

In various embodiments, a third subset of the first plurality of initial entity node encodings represent a second subset of the one or more proteins. For each initial entity node encoding in the third subset of the initial entity node encodings, the second attribute indicating the identity of the entity represented by the respective entity node comprises a modified amino acid sequence or an encoding of the modified amino acid sequence, and the one or more initial attributes of the respective initial entity node encoding include a third attribute indicating presence, absence, or expression level of the entity represented by the respective entity node.

In various embodiments, a fourth subset of the first plurality of initial entity node encodings represent the one or more regulators. For each initial entity node encoding in the fourth subset of the initial entity node encodings, the second attribute indicating the identity of the entity represented by the respective entity node comprises a nucleic acid sequence or an encoding of the nucleic acid sequence.

In various embodiments, a fifth subset of the first plurality of initial entity node encodings represent the one or more genes. For each initial entity node encoding in the fifth subset of the initial entity node encodings, the second attribute indicating the identity of the entity represented by the respective entity node comprises a nucleic acid sequence or an encoding of the nucleic acid sequence, and the one or more initial attributes of the respective initial entity node encoding include a third attribute indicating presence, absence, or expression level of the entity represented by the respective entity node.

In various embodiments, a sixth subset of the first plurality of initial entity node encodings represent the one or more transcripts. For each initial entity node encoding in the sixth subset of the initial entity node encodings, the second attribute indicating the identity of the entity represented by the respective entity node comprises a nucleic acid sequence or an encoding of the nucleic acid sequence, and the one or more initial attributes of the respective initial entity node encoding include a third attribute indicating presence, absence, or expression level of the entity represented by the respective entity node.

In various embodiments, a seventh subset of the first plurality of initial entity node encodings represent the one or more biomolecular complexes. For each initial entity node encoding in the seventh subset of the initial entity node encodings, the second attribute indicating the identity of the entity represented by the respective entity node comprises data identifying a neighborhood of other entity nodes representing one or more proteins, genes, regulators (e.g., regulatory DNA sequences), transcripts, and/or small molecules (e.g., metabolites).

In various embodiments, each initial entity node encoding in the plurality of initial entity node encodings corresponds to a respective entity node in the plurality of entity nodes and includes (i) a positional encoding of the respective entity node and/or (ii) a structural encoding of the respective entity node. In various embodiments, the positional encoding of the respective entity node encodes a local position of the respective entity node and/or a global position of the respective entity node. In various embodiments, the local position of the respective entity node comprises one or more measures of a position of the respective entity node within a subset of the graph, and the global position of the respective entity node comprises one or more measures of a position of the respective entity node within the graph.

In various embodiments, the subset of the graph comprises a local cluster of two or more nodes of the plurality of entity nodes and/or the plurality of interaction nodes. In various embodiments, the structural encoding of the respective entity node encodes a local substructure of the graph associated with the respective entity node and/or a global structure of the graph associated with the respective entity node. In various embodiments, each initial interaction node encoding in the second plurality of initial interaction node encodings corresponds to a respective interaction node in the second plurality of interaction nodes and includes (i) a positional encoding of the respective interaction node and/or (ii) a structural encoding of the respective interaction node.

In various embodiments, each initial edge encoding in the plurality of initial edge encodings corresponds to a respective directed edge in the plurality of directed edges and includes (i) a relative positional encoding of the respective directed edge and/or (ii) a relative structural encoding of the respective directed edge. The initial edge node encodings comprise vectors of a first length, the initial interaction node encodings comprise vectors of a second length, and the initial edge encodings comprise vectors of a third length.

In various embodiments, the one or more classes of the biological system include a tissue type of the biological system, and the one or more class encodings include a tissue type encoding representing the tissue type of the biological system. In various embodiments, the tissue type of the biological system comprises connective tissue, epithelial tissue, muscle tissue, and nervous tissue. In various embodiments, the tissue type encoding is generated using a transformer-based model pre-trained to perform masked language modeling (MLM) on biomedical literature. In various embodiments, the biological system prompts a pretrained model (e.g., another different machine learning model) to provide an encoding for a specific tissue type.

In various embodiments, the one or more classes of the biological system include a disease type of the biological system, and the one or more class encodings include a disease type encoding representing the disease type of the biological system. In various embodiments, the disease type of the biological system comprises an infectious disease, a deficiency disease, a hereditary disease (e.g., a genetic disease or non-genetic hereditary disease), and a physiological disease, optionally wherein the hereditary disease is a genetic disease, optionally wherein the genetic disease is cancer, optionally wherein the cancer is selected from bladder cancer, pancreatic cancer, cervical cancer, lung cancer, liver cancer, ovarian cancer, colon cancer, stomach cancer, virally induced cancer, neuroblastoma, breast cancer, prostate cancer, renal cancer, leukemia, sarcoma, carcinoma, non-small cell lung carcinoma, non-Hodgkin's lymphoma, acute myeloid leukemia (AML), chronic lymphocytic leukemia (CLL), B-cells chronic lymphocytic leukemia (B-CLL), multiple myeloma (MM), erythroleukemia, renal cell carcinoma, soft tissue sarcoma, melanoma, astrocytoma, oligoastrocytoma, bone cancer, brain cancer, gastrointestinal cancer, cardiac cancer, uterine cancer, head and neck cancer, gallbladder cancer, laryngeal cancer, lip and oral cavity cancer, ocular cancer, colorectal cancer, testicular cancer, throat cancer, acute lymphoblastic leukemia (ALL), chronic myelogenous leukemia (CML), adrenocortical carcinoma, AIDS-related lymphoma, primary CNS lymphoma, anal cancer. appendix cancer, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, extrahepatic ductal carcinoma in situ (DCIS), endometrial cancer, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, myelodysplastic syndromes, nasal cavity and paranasal sinus cancer, nasopharyngeal cancer, epithelial ovarian cancer, germ cell ovarian cancer, low malignant potential ovarian cancer, papillomatosis, paraganglioma, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, sezary syndrome, small intestine cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and waldenström macroglobulinemia.

In various embodiments, the disease type encoding is generated using a transformer-based model pre-trained to perform masked language modeling (MLM) on biomedical literature.

In various embodiments, the one or more classes of the biological system include a therapeutic agent applied to the biological system, and the one or more class encodings include a therapeutic agent encoding representing the therapeutic agent applied to the biological system. The therapeutic agent applied to the biological system comprises a small molecule, nucleic acid molecule, antibody or antibody fragment, or peptide. In various embodiments, the nucleic acid molecule is one of a vector, viral vector, short interfering RNA (siRNA), microRNA (miRNA), short hairpin RNA (shRNA), antisense oligonucleotide, nuclease, transposon, or aptamer.

In various embodiments, two or more of the initial entity node encodings correspond to two or more of the plurality of entity nodes representing two or more respective proteins. Each respective initial entity node encoding of the two or more initial entity node encodings comprises a respective binding vector including a plurality of binding scores corresponding to a plurality of small molecule drug compounds. Each binding score indicates a probability of the respective small molecule drug compound binding the respective protein. The therapeutic agent encoding for a specific small molecule drug compound comprises a feature vector converted from a SMILES string representing the specific small molecule drug compound.

In various embodiments, two or more entity nodes of the plurality of entity nodes represent two or more respective proteins. The therapeutic agent encoding for a specific small molecule drug compound comprises a binding vector including two or more binding scores corresponding to the two or more respective proteins. Each respective binding score indicates a probability of the specific small molecule drug compound binding the respective protein.

In various embodiments, each of the plurality of samples of the biological system belongs to a respective set of one or more of the classes of the biological system. Each of the plurality of records of the sample data indicates the set of one or more classes to which the respective sample of the biological system belongs. In various embodiments, each of the plurality of records of the sample data identify one or more proteins, one or more transcripts, and/or one or more genes present in the respective sample of the biological system. In various embodiments, each of the plurality of records of the sample data indicates expression levels of one or more proteins, one or more transcripts, and/or one or more genes in the respective sample of the biological system.

In various embodiments, the training includes progressively transforming the initial architectural encoding based on the training set of the sample data to produce an updated architectural encoding including a first plurality of updated entity node encodings corresponding, respectively, to the first plurality of entity nodes.

In various embodiments, the machine learning model comprises one or more neural networks (NNs). In various emblements, the machine learning model comprises one or more graph neural networks (GNNs). In various embodiments, the graph governs dataflow through the machine learning model to each of the one or more GNNs. In various embodiments, the one or more GNNs comprise one or more homogeneous GNNs. In various embodiments, at least one of the GNNs comprises a message passing layer (MPL) configured to pass information regarding at least a subset of the plurality of interactions from a first subset of the entities to a second subset of the entities.

In various embodiments, the one or more GNNs comprise a first heterogeneous GNN configured to generate a first subset of the updated entity node encodings corresponding to a first subset of the entity nodes representing a first subset of the one or more proteins. In various embodiments, the one or more GNNs comprise a second heterogeneous GNN configured to generate a second subset of the updated entity node encodings corresponding to a second subset of the entity nodes representing the one or more small molecules. In various embodiments, the one or more GNNs comprise a third heterogeneous GNN configured to generate a third subset of the updated entity node encodings corresponding to a third subset of the entity nodes representing a second subset of the one or more proteins. In various embodiments, the one or more GNNs comprise a fourth heterogeneous GNN configured to generate a fourth subset of the updated entity node encodings corresponding to a fourth subset of the entity nodes representing the one or more regulators. In various embodiments, the one or more GNNs comprise a fifth heterogeneous GNN configured to generate a fifth subset of the updated entity node encodings corresponding to a fifth subset of the entity nodes representing the one or more genes. In various embodiments, the one or more GNNs comprise a sixth heterogeneous GNN configured to generate a sixth subset of the updated entity node encodings corresponding to a sixth subset of the entity nodes representing the one or more transcripts.

In various embodiments, the training further includes decoding a subset of the updated entity node encodings representing a respective subset of the entities. The decoding includes generating predicted expression levels for the subset of the entities, determining a value of an error metric based on respective differences between the predicted expression levels and the measured expression levels for the subset of the entities; and adjusting a plurality of internal weights of the one or more GNNs to reduce the value of the error metric. The training set of the sample data include measured expression levels for the subset of the entities.

In various embodiments, training the model to model the biological system comprises training the model to predict expression levels of one or more first genes, transcripts, and/or proteins in a sample of the biological system based on input data indicating (i) one or more classes to which the sample of the biological system belongs and (ii) presence, absence, or expression levels of one or more second genes, transcripts, and/or proteins in the sample of the biological system.

In various embodiments, training the model to model the biological system further comprises training the model to classify the sample of the biological system as healthy or diseased based on the predicted expression levels of the one or more first genes, transcripts, and/or proteins and on the indicated presence, absence, or expression levels of one or more second genes, transcripts, and/or proteins in the sample of the biological system.

In various embodiments, training the model to model the biological system comprises training the model to determine one or more mechanisms of action of the biological system, to determine one or more pharmacokinetic properties of at least one entity of the biological system, and/or to determine one or more pharmacodynamic properties of at least one entity of the biological system.

In various embodiments, the biological system model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient descent. and dimensionality reduction techniques such as manifold learning, principal component analysis, factor analysis, autoencoder regularization, and independent component analysis, or combinations thereof. In particular embodiments, the biological system model is trained using a deep learning algorithm. In particular embodiments, the biological system model is trained using a random forest algorithm. In particular embodiments, the biological system model is trained using a linear regression algorithm. In various embodiments, the biological system model is trained using supervised learning algorithms, unsupervised learning algorithms, semi-supervised learning algorithms (e.g., partial supervision), weak supervision, transfer, multi-task learning, or any combination thereof. In particular embodiments, the biological system model is trained using supervised learning algorithms. In various embodiments, the biological system model is trained using one or more of the above described algorithms.

In various embodiments, the biological system model can be trained in multiple different stages, where each stage may use a same or different types of data for the respective training. In one example, the biological system model can be first trained with structural and positional related data, The biological system model trained in this way may better capture the structural and/or positional information of the nodes in the biological system graph. For example, the biological system model may include a neural network for structural and positional encoding, which can be trained with the structural and positional related data. In a next stage, the biological system model may be trained with omics data, such as proteomics data, genomics data, epigenetics data, metabolomics data, and/or transcriptomics data. The biological system model trained with these data may be used to predict the expression levels of certain proteins, transcripts, or genes. In some embodiments, the biological system model trained with the omics data can be further trained with a large variety of drugs. The biological system model trained with the drugs can be then used for drug screening, for example, based on the perturbation signatures identified from the other drugs during the training process. In some embodiments, the biological system model can be also trained with other types of data. and be used for other purposes not described in the present disclosure.

Referring now to FIG. 8, a process 800 for deploying a biological system model (e.g., using the biological system model to generate predictions) is further depicted, in accordance with one embodiment. The deployed biological system model can be any of the models described elsewhere herein. For example, the biological system model can be any of the models trained through the above described training process 700.

The process 800 may include step 802 of obtaining input sample data comprising a record derived from a first sample of a biological system. The record may indicate presence, absence, and/or expression levels of one or more entities in the first sample of the biological system, and one or more first classes (e.g., a biological system at a specific healthy, disease, or treated state from a specific tissue or organ) to which the first sample of the biological system belongs.

The process 800 may include step 804 of providing the input sample data as input to the machine learning model trained to model the biological system. The model may be trained as described above. For example, the model may be initialized using biological system data and trained using training sample data. The biological system data may include architectural data (G_{L1}) and class data, as described earlier. The architectural data may represent a bipartite graph representing the biological system. The graph may include a first plurality of entity nodes representing a plurality of entities included in the biological system, a second plurality of interaction nodes representing a plurality of interactions between respective subsets of the plurality of entities, and a plurality of directed edges connecting a plurality of node pairs. Each node pair may include a respective first node representing an entity of the plurality of entities and a respective second node representing an interaction of the plurality of interactions. The graph may be structured as a closed-loop control system (e.g., optimal control loop). The architectural data (G_{L1}) may include an architectural encoding including a first plurality of entity node encodings corresponding, respectively, to the first plurality of entity nodes. Each entity node encoding may indicate one or more attributes of the entity represented by the respective entity node. The architectural encoding may include a second plurality of interaction node encodings corresponding, respectively, to the second plurality of interaction nodes. Each interaction node encoding may indicate one or more attributes of the interaction represented by the respective interaction node. The architectural encoding may include a plurality of edge encodings corresponding, respectively, to the plurality of directed edges. Each edge encoding may indicate one or more attributes of the respective directed edge. The class data may include one or more class encodings representing one or more respective classes of the biological system. Each class encoding may indicate one or more attributes of the respective class of the biological system. The training sample data may include a plurality of records derived from a respective plurality of second samples of the biological system, where each record indicates presence, absence, and/or expression levels of one or more of the plurality of entities in the respective second sample of the biological system.

The process 800 may include step 806 of determining one or more attributes of the biological system based on the output of the machine learning model. For example, once trained, the model may be used to determine presence, absence, and/or expression levels of one or more of the plurality of entities in the first sample of the biological system. In another example, the trained model may be used to classify the first sample as healthy or diseased based on the determined presence, absence, and/or expression levels of one or more of the plurality of entities in the first sample of the biological system. In yet another example, the trained model may be used to determining one or more mechanisms of action in the first sample of the biological system, and/or determining one or more pharmacokinetic and/or pharmacodynamic properties of the first sample of the biological system. In yet another example, the trained model may be used to determine one or more attributes of the first sample of the biological system comprises determining one or more second classes to which the first sample of the biological system belongs. In yet another example, the trained model may be used to a presence of cytotoxicity, growth inhibition, and/or apoptosis in the first sample of the biological system.

In various embodiments, the input sample data described above may further include other necessary information that may be input into the trained model. For example, if the first sample is found to be a first tissue type, a first tissue type encoding may be obtained and provided as the input to the trained model. In another example, if the first sample is found to be a first disease type, a first disease type encoding may be obtained and provided as the input to the trained model. In yet another example, if the first sample is found to treated with a first therapeutic agent, a first therapeutic agent encoding may be obtained and provided as the input to the trained model. Other information may be also input to facilitate the trained model to determine one or more attributes of the biological system.

In some embodiments, when properly configured and trained, the model may be used for other purposes not described above. For example, by taking into consideration of SNPs in the model development and training process, the model may be used to predict drug efficacy for a patient with specific SNPs. For example, a drug having been found to be effective on other patients may be ineffective on a specific patient due to the unique SNPs of the patient, which affects the efficacy of the drug on the patient. For this reason, a healthcare provider may be equipped with a system containing a trained machine learning model disclosed herein, which can be used to predict whether a drug is effective for that specific patient by providing the SNP information of that patient to the model.

### System and/or Computer-Based Embodiments

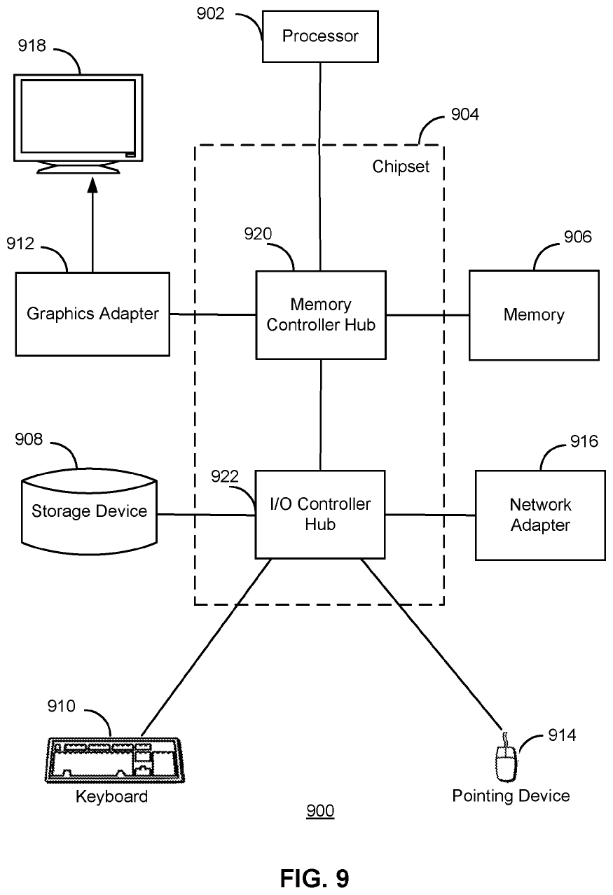

FIG. 9 depicts an example computing device 900 for implementing the systems and methods described in reference to FIGS. 1-8. Examples of a computing device can include a personal computer, desktop computer laptop, server computer, a computing node within a cluster, message processors, hand-held devices, multi-processor systems. microprocessor-based or programmable consumer electronics, network PCs, minicomputers, mainframe computers, mobile telephones, PDAs, tablets, pagers, routers, switches, and the like. In various embodiments, the computing device 900 can operate as the predictive model 130 (or a portion of the predictive model 130) shown in FIG. 1. Thus, the computing device 900 may train and/or deploy predictive models for predicting potential drugs for certain diseases.

In some embodiments, the computing device 900 includes at least one processor 902 coupled to a chipset 904. The chipset 904 includes a memory controller hub 920 and an input/output (I/O) controller hub 922. A memory 906 and a graphics adapter 912 are coupled to the memory controller hub 920, and a display 918 is coupled to the graphics adapter 912. A storage device 908, an input interface 914, and a network adapter 916 are coupled to the I/O controller hub 922. Other embodiments of the computing device 900 have different architectures.

The storage device 908 is a non-transitory computer-readable storage medium such as a hard drive, compact disk read-only memory (CD-ROM), DVD, or a solid-state memory device. Memory 906 holds instructions and data used by processor 902. The input interface 914 is a touch-screen interface, a mouse, track ball, or other type of input interface, a keyboard 910, or some combination thereof, and is used to input data into the computing device 900. In some embodiments, the computing device 900 may be configured to receive input (e.g., commands) from the input interface 914 via gestures from the user. The graphics adapter 912 displays images and other information on the display 918. The network adapter 916 couples the computing device 900 to one or more computer networks.

The computing device 900 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 908, loaded into the memory 906, and executed by the processor 902.

The types of computing devices 900 can vary from the embodiments described herein. For example, the computing device 900 can lack some of the components described above, such as graphics adapters 912, input interface 914, and displays 918. In some embodiments, a computing device 900 can include a processor 902 for executing instructions stored on a memory 906.

The methods disclosed herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage. e.g., word processing text file, database format, etc.

### Additional Embodiments and Case Studies

In some embodiments, a biological system model may be trained using omics data from samples taken from a wide variety of human subjects. In such cases, the genomic information in the model may represent a ‘generic’ human genome (e.g., an average or probable genome for the human population). In some embodiments, a biological system model may be trained using omics data from samples taken from an individual human subject or a genetically related group of human subjects. In such cases, the genetic information in the model may represent a ‘personal’ or ‘familial’ human genome. Such a model may be particularly suited for applications in personalized medicine.

In some embodiments, a biological system model may be pre-trained using omics data from samples taken from a wide variety of human subjects (to generate a pre-trained model with genomic information representing a generic human genome), and then further trained using omics data from samples taken from an individual human subject or a genetically related group of human subjects (to customize the model to the individual or family for applications in personalized medicine).

More generally, in some embodiments, a biological system model may be trained in two stages. In the first stage, the model may be trained using omics data from a wide variety of samples (e.g., samples of a variety of different tissues, samples with a variety of different diseases, samples treated with a variety of different therapeutic agents, samples taken from a wide variety of people, etc.). The model generated by the first stage of training may be a general model of the biological system, suitable for general use. In the second stage, the model may be trained using omics data from samples sharing specific attributes (e.g., a specific tissue type, genome, disease, etc.). The model generated by the second stage of training may be a customized model of the biological system, particularly well-suited for use in modeling instances of the biological system having those specific attribute(s).

Examples have been described in which a biological system model of a human cell is generated. In some embodiments, models of cells of other organisms (e.g., other animals) may be generated.

In some embodiments, the transcriptomic data includes post-translational modifications.

Omics data obtained from different sources (e.g., different databases) may be reported in different units. In some embodiments, before omics data are encoded in the model or used to train the model, the data may be normalized so that all expression levels of the same type are reported in the same units (e.g., transcripts per million or log-fold change).

Examples have been described in which the inputs to the model and/or outputs of the model may indicate the presence, absence, or expression level of transcripts, proteins, small molecules (metabolites), or other observables. The presence or absence of an observable may be represented by a binary value (e.g., 0 or 1). The expression level of any observable may be represented by a numeric value (e.g., an integer or real number).

Examples have been described in which binding scores indicate the probability of specific drugs binding specific proteins represented by entity nodes in the biological system graph. In some embodiments, the binding scores may be provided to a classifier, which may provide output predicting whether the protein is bound or not bound by the drug, and these binary classifications (bound vs. not bound) may be used in the level-2 model.

In some embodiments, sample data used to build and train a biological system model may be drawn from any suitable cell line or set of cell lines, including (without limitation) A375, A549, A673, AGS, BT20, CL34, CORL23, COV644, DV90, EFO27, H1299, HA1E, HA1E.101, HCC15, HCC515, HCT116, HEC108, HEK293T, HEKTE, HELA, HEPG2, HL60, HME1, HS27A, HS578T, HT115, HT29, HUH7, HUVEC, JHUEM2, JURKAT, LNCAP, LOVO, MCF10A, MCF7, MCF7.101, MCH58, MDAMB231, MDST8, NCIH1694, NCIH1836, NCIH2073, NCIH508, NCIH596, NCIH716, NKDBA, NOMO1, OV7, PC3, PC3.101, PL21, RKO, RMGI, RMUGS, SHSYSY, SKBR3, SKLU1, SKM1, SKMEL1,SKMEL28, SNGM, SNU1040, SNUC4, SNUC5, SW480, SW620, SW948, T3M10, THP1, TYKNU, U266, U2OS, U937, VCAP, WSUDLCL2, YAPC, MNEU.E. NEU, NEU.KCL, NPC, NPC.CAS9, NPC.TAK, HUES3, FIBRNPC, ASC, ASC.C, CD34, PHH, SKB, SKL, SKL.C, 22Rv1, BE2C, C4-2B, GM08714, GM12878, GM12891, GM12892, GM23338, H1, H54, HEK293, HeLa-S3, IMR-90, Ishikawa, K562, MM.1S, NB4, NT2/D1, OCI-LY3, PC-9, Panc1, RWPE1, SK-N-SH, and/or T47D (from LINCS data).

### A Case Study of Non-Small Cell Lung Carcinoma in Lung

In actual applications, characterizing changes in protein and transcript levels in a cell or tissue in the disease state compared to the healthy state allows a user to determine which mechanisms are disrupted in a disease. Identifying perturbed mechanisms allows a drug discovery scientist to identify potential new drug targets or devise an assay strategy to validate these drug targets. When using different data modalities (e.g., proteins and transcripts) as inputs to machine learning models in characterizing changes in protein and transcript levels, it is important to employ an approach that accounts for their limitations in representing the state of the cell.

Proteins are the main effectors of signaling cascades, processes, and regulatory interactions within a cell, but it is generally easier to measure transcripts than proteins. Both data modalities tend to be noisy due to technical artifacts and heterogeneity in gene expression in a given sample. Proteomics data tend to be sparse, but using transcriptomics to quantify gene expression in order to account for this sparsity is challenging. In a given cell or tissue, transcript levels are moderately correlated with the levels of their corresponding translated proteins due to differences in the rates of transcription/translation and degradation of the two types of molecules. Furthermore, protein abundance is not the only driver of the change in the state of the cell, as proteins are also modified after translation, resulting in a change in function or location. Due to these limitations, some embodiments of the simulation pipeline disclosed herein takes a multi-omics approach to understanding how transcript levels influence protein levels and vice versa. In the following, a case study is further provided.

Non-small cell lung carcinoma (NSCLC) is a disease indication with a high unmet need. To identify targeted therapies that are more effective and less toxic than currently available chemotherapeutic agents, the molecular mechanisms of the disease need to be better understood.

Towards this objective, some embodiments of the model (e.g., graph neural network for cellular behavior simulation) disclosed herein may be trained on omics data (e.g., transcriptomics and/or proteomics data) from healthy and diseased lung tissue. In the healthy state and disease state, transcript levels were present for 54,638 and 45,601 nodes out of the total 134,741 transcript nodes, respectively. Of the total 94,192 protein nodes in the biological system graph, protein levels were present for 5,508 nodes and 10,195 nodes in the healthy and disease states, respectively.

During the training process, the values for twenty percent of the labeled nodes (i.e., the nodes with data available) were held out from the model as the validation set. The model performance was then evaluated using a number of metrics, including Pearson's correlation coefficient (PCC) between the predicted and actual values in the validation set.

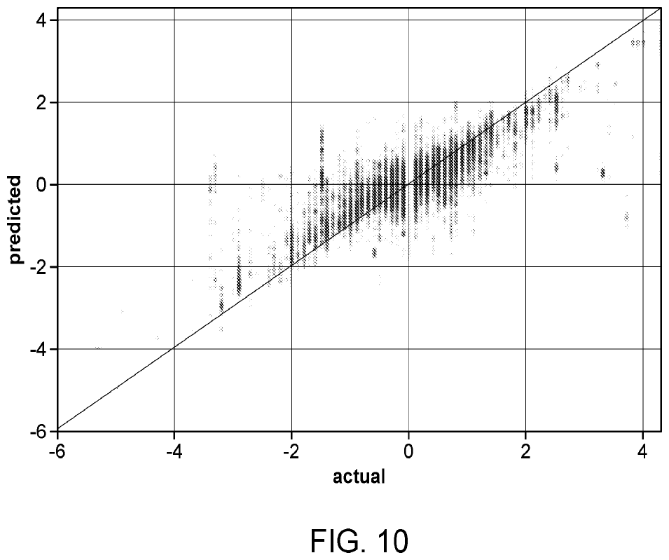

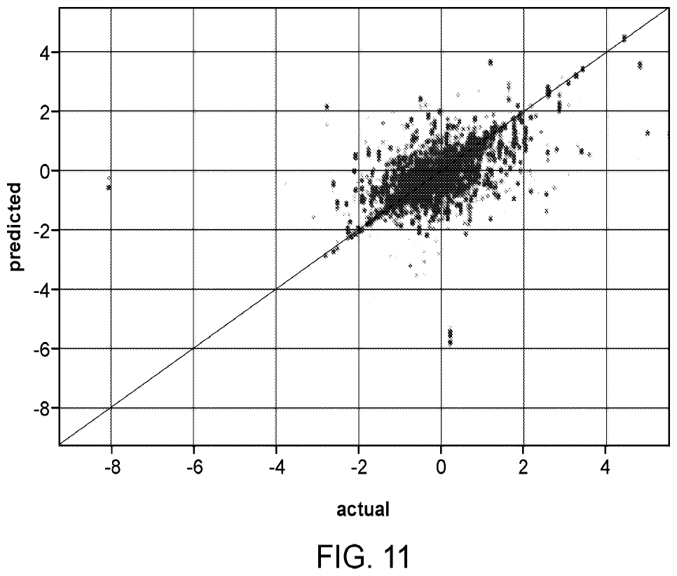

After training the model with eighty percent of the data, the model was tested using the remaining 20% of the data for model validation. In the disease state, the PCC for transcript levels predicted by the model compared to the actual of the log₂-fold of transcript levels in NSCLC in the validation set was 0.83, as shown in FIG. 10. The performance of the trained model shows a great improvement when compared to other known models for predicting transcript levels. The PCC for normalized protein levels in the disease state in NSCLC predicted by the trained model compared to actual protein levels in the validation set was 0.65, as shown in FIG. 11. This also indicates a good performance of the trained model in predicting protein levels.

### Drug-Target Interaction Embeddings

In actual applications, drugs affect the state of the system predominantly through drug-target interactions, where the target is a protein and a drug affects protein function by binding to it. The protein's function can then be inhibited or activated by the drug. To determine the source of small molecule perturbation on the omics graph through additional input embedding in the treated model, a prediction of drug-target interaction is necessary. Many existing models use a dual transformer approach which separately embeds a drug molecule SMILES string and a protein amino acid sequence, then combines the embedded representations to form a prediction of the interaction.

In the model disclosed herein, a dual transformer model was developed to classify a drug/target combination as bound or not bound. The model hyperparameters that best predict the mechanism of action of a drug according to the library of integrated network-based cellular signatures (LINCS) L1000 dataset were then selected and used to generalize the top-ranked proteins (e.g., top 5, top 10, top 15, top 20, top 25, top 30, etc.) across all proteins in the biological system graph. Furthermore, inferences were performed with this model to build a full matrix of binding embeddings across a large number (e.g., 10 k, 15 k, 20 k, 25 k, etc.) of small molecules and all the proteins (e.g., around 90-100 k proteins). These binding embeddings were then concatenated with existing protein node features in the implementation of the treated model.

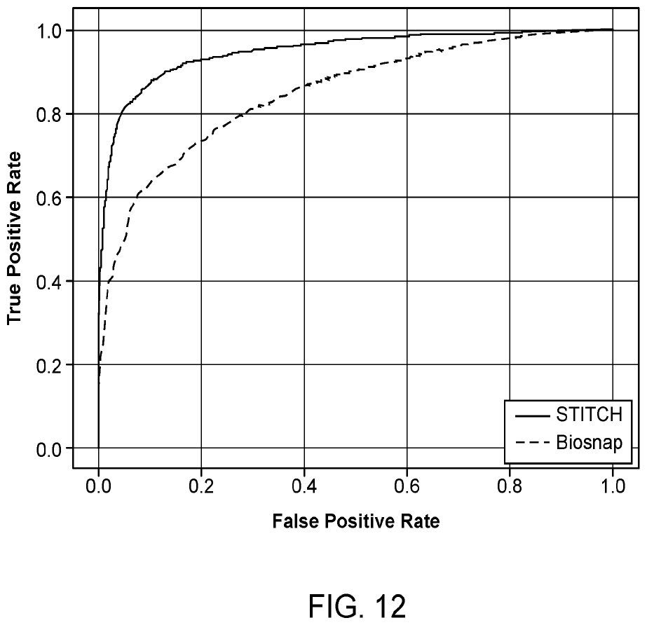

FIG. 12 illustrates receiver operating characteristic (ROC) curves obtained from predicting bound or not-bound drug-target pairs by using the binding model (e.g., dual transformer model) disclosed herein. In FIG. 12, two different datasets STITCH and BioSNAP were used to train the dual transformer model. The dataset used for testing was a set of LINCS L1000 mechanism of action drug-target pairs. From the figure, it can be seen that both the ROC curves are much closer to the top left corner, which indicates that the binding model disclosed herein may effectively identify the targets for a drug.

The following is a table (Table-1) that summarizes the performance of LINCS MoA prediction using different data sources.

TABLE 1
Performance of LINCS MoA prediction
using different data sources.

| Task | Position | Score (Val) |
|---|---|---|
| LINCS MoA Prediction | SotA (State-of-the-art) | 0.85 AUROC |
| (BioSnap) | | |
| LINCS MoA Prediction | SotA | 0.945 AUROC |
| (STITCH) | | |

The following is another table (Table-2) that summarizes the mechanism of action recall for six EGFR inhibitors including erlotinib, GW-583340, canertinib, gefitinib, lapatinib, and AG-957. In the table, the mechanism of action (MoA) means the specific targets in a cell predicted by the model (also referred to as “predicted treated perturbation signature”), and the rank means that the corresponding rank of that target when compared to other targets predicted for that inhibitor. As can be seen from the table, based on the predicted treated perturbation signatures, the platform was largely able to recall the known mechanisms of action for EGFR inhibitors. This finding suggests that the model is able to characterize the response of the cell to the drug based on the predicted treated perturbation signature.

TABLE 2
Mechanism of Action Recall for EGFR Inhibitors.

| Drug | Mechanism of Action | Rank |
|---|---|---|
| Erlotinib | EGFR | 2 |
| Lapatinib | EGF4 | 4 |
| | ERBB2 | >20 |
| | EGFR | 12 |
| GW583340 | ERBB2 | 1 |
| Canertinib | EGFR | 1 |
| | ERBB2 | 2 |
| | ERBB4 | 3 |
| Gefitinib | EGFR | 13 |
| AG-957 | EGFR | 3 |
| | BCR/ABL | 4 (ABL) |

In some embodiments, the treated model may be also used to predict transcript or protein levels for unseen transcripts or proteins as described above for the healthy model or disease model. For this purpose, the treated model was first trained with transcriptomics data following small molecule perturbation. In the first stage, the model was trained to overfit on a specific disease-tissue-drug combination in order to predict transcript levels for unseen transcripts in the validation set as described above. That is, the model was trained with data for a specific disease-tissue-drug combination. The trained model can be then used to predict the transcript or protein levels for unseen transcripts or proteins for the samples in a same combination.

In the second stage, the treated model was further trained to generalize across drug molecules to predict transcriptome-wide changes for an unseen molecule (e.g., a molecule that has not performed any perturbation study yet). That is, a lot of different drugs (e.g., various drugs known to be effective to treat a disease) were used to train a same treated model. These different drugs may cover various mechanisms of action in the treatment. After training the treated model as described above, the trained treated model can be then used to predict whether a “unseen” (i.e., not tested previously) molecule is effective in treating the disease, based on the predicted transcriptome-wide changes for the unseen molecule. In the following, a specific case study is further described, which is related to a treated model after the first stage of training.

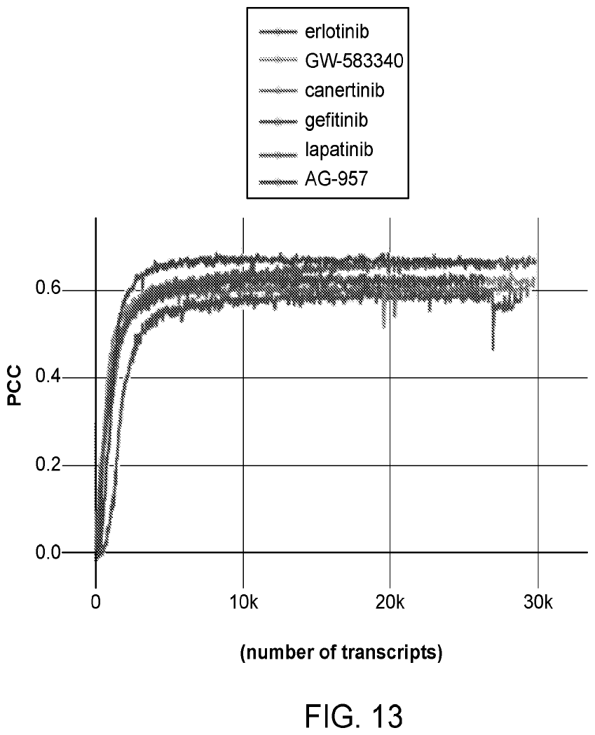

EGFR inhibitors are a commonly prescribed targeted therapy in non-small cell lung carcinoma. The training data for these simulations were generally more sparse than for the simulations performed in the disease state. On average, transcript levels were available for about 28,000 transcript nodes out of 134,741 total transcript nodes in the graph. However, despite this sparsity, the model was able to achieve a PCC of 0.613 to 0.691, as shown in FIG. 13, between actual and predicted transcript levels in the validation set for six EGFR inhibitors, including erlotinib, GW-583340, canertinib, gefitinib, lapatinib, and AG-957. The PCC values indicate that the treated model can be effective in predicting transcript or protein levels for unseen transcripts or proteins.

In the following table (Table-3), the performance scores of the disclosed various models are further provided.

TABLE 3
Performance across tasks in the transductive setting.

| Task | Position | Score (Val) |
|---|---|---|
| Transcript (Health) | SotA | 0.79 (r) |
| Transcript (Disease) | SotA | 0.75-0.83 (r) |
| Transcript (Treated) | SotA | up to 0.74 (r) |
| Protein (Health) | SotA | 0.49-0.66 (r) |
| Protein (Disease) | SotA | 0.47-0.64 (r) |
| Protein (Treated) | n/a | n/a |

In the table, the units are as follows: Health State: Correlation Score (TPM), Disease State: Correlation Score (Log₂Change), and Treated State: Correlation Score (Log₂Change). From these scores, it can be seen that the disclosed model(s) including different disease, healthy, and treated models performed better than other existing approaches in transcript or protein expression level prediction.

### Predicting Cytotoxicity

In some embodiments, the model disclosed herein may be also used to predict the cytotoxicity of a drug compound or another different treatment agent. When testing the effect of a compound on cells in culture, IC50, the most commonly used drug response metric, usually refers to the molar concentration of a drug that is needed to inhibit a biological process, pathway or activity by 50%. However, when testing the ability of a compound to inhibit the growth of cells in culture, GI50 is preferred. GI50, or the “growth inhibition” concentration, refers to the molar concentration of a compound needed to reduce the growth of cells in culture by 50%. Because chemotherapy is intended to be cytotoxic to tumor cells, GI50 can be used as a measure of the efficacy of a potential new chemotherapeutic agent.

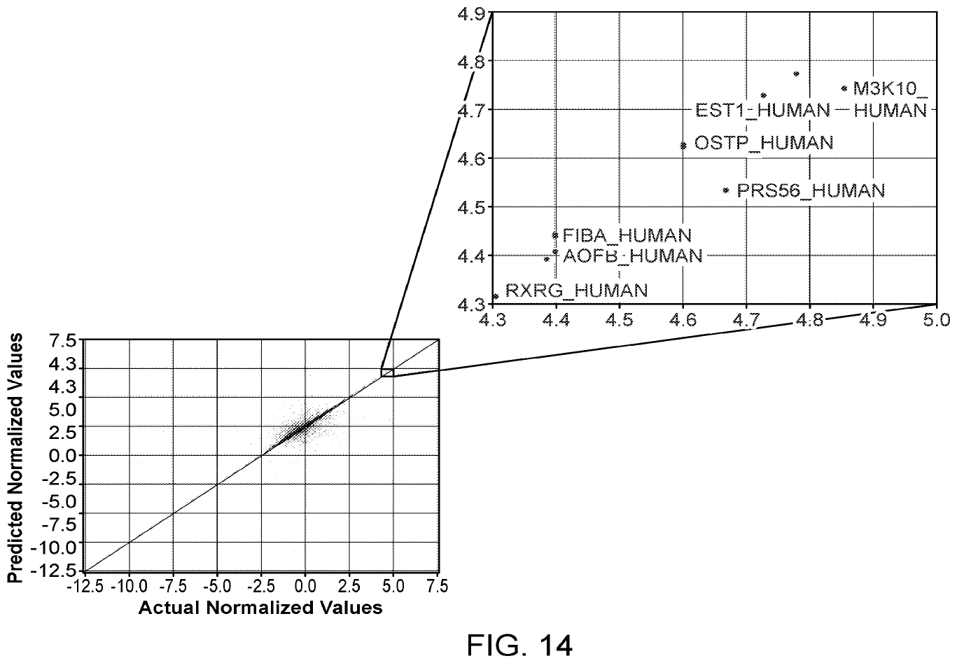

To predict the efficacy of a drug compound in cancer cell lines, the model was trained to predict GI50 using dose-response curves from publicly available data sources such as the genomics of drug sensitivity in cancer (GDSC). After the training, the trained treated model was then used to predict the cytotoxicity of an unseen molecule. FIG. 14 predicted protein levels and actual protein levels in lung cancer (NSCLC) tissue using a trained disease model. In the main scatter plot and zoom-in plot, the dots represent different cell lines. As can be seen from the two plots, the trained treated model can predict the cytotoxicity of a drug with a high confidence index.

In the foregoing embodiments, a novel, principled approach to modeling a biological system in the presence of a disease and a drug is disclosed. Briefly, the cellular dynamics were modeled in the form of an optimal control loop with the key interactions being those between the genes, transcripts, and proteins. The connectivity between these entities was enforced through the use of a directed graph. In typical formulations of an optimal control system, it is necessary to have a symbolic representation for the interactions, or coupling, between the different components in the system that represent the state, actuators, sensors, and feedback. However, a symbolic representation for these interactions is generally unknown, as is the case with biological systems in general. To circumvent this, the data-driven paradigm of representing the interactions was employed using message-passing neural networks, where the interactions were learned via supervised machine learning. The supervised training at a node level for the transcript and protein nodes was performed using a single dataset that is comprised of publicly-available and privately-provided datasets that were collected using different experimental techniques. Since the data-driven representations of the dynamics were used, it was able to get a single, unified model for predicting disparate observables.

Various embodiments are described and illustrated in this specification to provide an overall understanding of the composition, function, operation, and application of the disclosed compositions and methods. It is understood that the various embodiments described and illustrated in this specification are non-limiting and non-exhaustive. Thus, the invention is not necessarily limited by the description of the various non-limiting and non-exhaustive embodiments disclosed in this specification. The features and characteristics illustrated or described in connection with various embodiments may be combined with the features and characteristics of other embodiments. Such modifications and variations are intended to be included within the scope of this specification. As such, the claims may be amended to recite any features or characteristics expressly or inherently described in, or otherwise expressly or inherently supported by this specification. Further, Applicant reserves the right to amend the claims to affirmatively disclaim features or characteristics that may be present in the prior art. The various embodiments disclosed and described in this specification can comprise, include, consist of, or consist essentially of the features and characteristics as variously described in this specification.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

The use of certain terms in various places in the specification is for illustration purposes only and should not be construed as limiting. A service, function, or resource is not limited to a single service, function, or resource; usage of these terms may refer to a grouping of related services, functions, or resources, which may be distributed or aggregated.

Furthermore, connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

Furthermore, regarding the methods described herein, one skilled in the art would appreciate that (1) certain steps may optionally be performed; (2) steps may not be limited to the specific order set forth herein; (3) certain steps may be performed in different orders; and (4) certain steps may be performed simultaneously or concurrently.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion. i.e., “one or more” of the elements so conjoined. Other elements may optionally be present other than the elements specifically identified by the “and/or” clause, whether related or unrelated to those elements specifically identified. Thus, as a non-limiting example. a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally. additional unlisted items. Only terms clearly indicated to the contrary, such as “only one of or “exactly one of,” or, when used in the claims. “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

1. A method for generating a model of a biological system, the method comprising:
obtaining biological system data including architectural data (G_{L1}) and class data,
wherein the architectural data represent a bipartite graph representing a biological system, wherein (i) the graph includes a first plurality of entity nodes representing a plurality of entities included in the biological system, a second plurality of interaction nodes representing a plurality of interactions between respective subsets of the entities, and a plurality of edges connecting a plurality of node pairs, each node pair including a respective first node representing an entity of the plurality of entities and a respective second node representing an interaction of the plurality of interactions, (ii) the graph is structured as a closed-loop control system, and (iii) the architectural data (G_{L1}) include an initial architectural encoding including a first plurality of initial entity node encodings corresponding, respectively, to the first plurality of entity nodes, each initial entity node encoding indicating one or more initial attributes of the entity represented by the respective entity node, a second plurality of initial interaction node encodings corresponding, respectively, to the second plurality of interaction nodes, each initial interaction node encoding indicating one or more initial attributes of the interaction represented by the respective interaction node, and a plurality of initial edge encodings corresponding, respectively, to the plurality of edges, each initial edge encoding indicating one or more initial attributes of the respective edge, and
wherein the class data include one or more class encodings representing one or more respective classes of the biological system, each class encoding indicating one or more attributes of the respective class of the biological system; and
obtaining sample data comprising a plurality of records derived from a respective plurality of samples of the biological system, each record indicating presence, absence, and/or expression levels of one or more of the entities in the respective sample of the biological system;
dividing the sample data into a training set and a validation set;
providing the biological system data as input to a machine learning model to initialize the machine learning model;
training the model to model the biological system based on the training set of the sample data; and
validating the trained model using the validation set of the sample data.

2. (canceled)

3. The method of claim 1, wherein the plurality of entities include one or more proteins, one or more genes, one or more transcripts, one or more small molecules, one or more biomolecular complexes, and/or one or more regulators, and wherein the plurality of interactions include one or more biochemical reactions, one or more transcription events, one or more translation events, one or more physical regulations, one or more indirect regulations, one or more degradations, one or more genomic connections, and/or one or more pathway.

4-28. (canceled)

29. The method of claim 3, wherein for each initial entity node encoding in the first plurality of initial entity node encodings, the one or more initial attributes of the respective initial entity node encoding include a first attribute indicating an entity type of the entity represented by the respective entity node, and wherein the entity type is a protein, gene, transcript, small molecule, biomolecular complex, modified protein, or regulator.

30. The method of claim 3, wherein for each initial entity node encoding in the first plurality of initial entity node encodings, the one or more initial attributes of the respective initial entity node encoding include a second attribute indicating an identity of the entity represented by the respective entity node.

31. The method of claim 30, wherein:
a first subset of the first plurality of initial entity node encodings represent a first subset of the one or more proteins; and
for each initial entity node encoding in the first subset of the initial entity node encodings,
the second attribute indicating the identity of the entity represented by the respective entity node comprises an amino acid sequence or an encoding of the amino acid sequence, and
the one or more initial attributes of the respective initial entity node encoding include a third attribute indicating presence, absence, or expression level of the entity represented by the respective entity node.

32. (canceled)

33. The method of claim 30, wherein:
a second subset of the first plurality of initial entity node encodings represent the one or more metabolites; and
for each initial entity node encoding in the second subset of the initial entity node encodings, the second attribute indicating the identity of the entity represented by the respective entity node comprises a set of Simplified Molecular Input Line Entry System (SMILES) strings or an encoding of the set of SMILES strings.

34. The method of claim 30, wherein:
a third subset of the first plurality of initial entity node encodings represent a second subset of the one or more proteins; and
for each initial entity node encoding in the third subset of the initial entity node encodings,
the second attribute indicating the identity of the entity represented by the respective entity node comprises a modified amino acid sequence or an encoding of the modified amino acid sequence, and
the one or more initial attributes of the respective initial entity node encoding include a third attribute indicating presence, absence, or expression level of the entity represented by the respective entity node.

35. The method of claim 30, wherein:
a fourth subset of the first plurality of initial entity node encodings represent the one or more regulators; and
for each initial entity node encoding in the fourth subset of the initial entity node encodings, the second attribute indicating the identity of the entity represented by the respective entity node comprises a nucleic acid sequence or an encoding of the nucleic acid sequence.

36. The method of claim 30, wherein:
a fifth subset of the first plurality of initial entity node encodings represent the one or more genes; and
for each initial entity node encoding in the fifth subset of the initial entity node encodings,
the second attribute indicating the identity of the entity represented by the respective entity node comprises a nucleic acid sequence or an encoding of the nucleic acid sequence, and
the one or more initial attributes of the respective initial entity node encoding include a third attribute indicating presence, absence, or expression level of the entity represented by the respective entity node.

37. The method of claim 30, wherein:
a sixth subset of the first plurality of initial entity node encodings represent the one or more transcripts; and
for each initial entity node encoding in the sixth subset of the initial entity node encodings,
the second attribute indicating the identity of the entity represented by the respective entity node comprises a nucleic acid sequence or an encoding of the nucleic acid sequence, and
the one or more initial attributes of the respective initial entity node encoding include a third attribute indicating presence, absence, or expression level of the entity represented by the respective entity node.

38. (canceled)

39. The method of claim 30, wherein:
a seventh subset of the first plurality of initial entity node encodings represent the one or more biomolecular complexes; and
for each initial entity node encoding in the seventh subset of the initial entity node encodings, the second attribute indicating the identity of the entity represented by the respective entity node comprises data identifying a neighborhood of other entity nodes representing one or more proteins, genes, regulators, transcripts, and/or small molecules.

40. The method of claim 1, wherein each initial entity node encoding in the plurality of initial entity node encodings corresponds to a respective entity node in the plurality of entity nodes and includes (i) a positional encoding of the respective entity node and/or (ii) a structural encoding of the respective entity node.

41-44. (canceled)

45. The method of claim 1, wherein each initial interaction node encoding in the second plurality of initial interaction node encodings corresponds to a respective interaction node in the second plurality of interaction nodes and includes (i) a positional encoding of the respective interaction node and/or (ii) a structural encoding of the respective interaction node.

46. The method of claim 1, wherein each initial edge encoding in the plurality of initial edge encodings corresponds to a respective directed edge in the plurality of edges and includes (i) a relative positional encoding of the respective edge and/or (ii) a relative structural encoding of the respective edge.

47. The method of claim 1, wherein the initial edge node encodings comprise vectors of a first length, the initial interaction node encodings comprise vectors of a second length, and the initial edge encodings comprise vectors of a third length.

48. The method of claim 1, wherein the one or more classes of the biological system include a tissue type of the biological system, and wherein the one or more class encodings include a tissue type encoding representing the tissue type of the biological system.

49-50. (canceled)

51. The method of claim 1, wherein the one or more classes of the biological system include a disease type of the biological system, and wherein the one or more class encodings include a disease type encoding representing the disease type of the biological system.

52-53. (canceled)

54. The method of claim 1, wherein the one or more classes of the biological system include a therapeutic agent applied to the biological system, and wherein the one or more class encodings include a therapeutic agent encoding representing the therapeutic agent applied to the biological system.

55-58. (canceled)

59. The method of claim 3, wherein each of the plurality of samples of the biological system belongs to a respective set of one or more of the classes of the biological system.

60-62. (canceled)

63. The method of claim 3, wherein the training includes progressively transforming the initial architectural encoding based on the training set of the sample data to produce an updated architectural encoding including a first plurality of updated entity node encodings corresponding, respectively, to the first plurality of entity nodes.

64-75. (canceled)

76. The method of claim 1, wherein training the model to model the biological system comprises training the model to predict expression levels of one or more first genes, transcripts, and/or proteins in a sample of the biological system based on input data indicating (i) one or more classes to which the sample of the biological system belongs and (ii) presence, absence, or expression levels of one or more second genes, transcripts, and/or proteins in the sample of the biological system.

77. (canceled)

78. The method of claim 1, wherein training the model to model the biological system comprises training the model to simulate dynamic behavior of the biological system, to determine one or more mechanisms of action of the biological system, to determine one or more pharmacokinetic properties of at least one entity of the biological system, and/or to determine one or more pharmacodynamic properties of at least one entity of the biological system.

79. A biological system modeling method, comprising:
obtaining input sample data comprising a record derived from a first sample of a biological system, the record indicating (i) presence, absence, and/or expression levels of one or more entities in the first sample of the biological system, and (ii) one or more first classes to which the first sample of the biological system belongs;
providing the input sample data as input to a machine learning model trained to model the biological system, wherein
the machine learning model has been initialized using biological system data and trained using training sample data,
the biological system data include architectural data (G_{L1}) and class data,
the architectural data represent a bipartite graph representing the biological system, wherein (i) the graph includes a first plurality of entity nodes representing a plurality of entities included in the biological system, a second plurality of interaction nodes representing a plurality of interactions between respective subsets of the plurality of entities, and a plurality of edges connecting a plurality of node pairs, each node pair including a respective first node representing an entity of the plurality of entities and a respective second node representing an interaction of the plurality of interactions, (ii) the graph is structured as a closed-loop control system, and (iii) the architectural data (G_{L1}) include an architectural encoding including a first plurality of entity node encodings corresponding, respectively, to the first plurality of entity nodes, each entity node encoding indicating one or more attributes of the entity represented by the respective entity node, a second plurality of interaction node encodings corresponding, respectively, to the second plurality of interaction nodes, each interaction node encoding indicating one or more attributes of the interaction represented by the respective interaction node, and a plurality of edge encodings corresponding, respectively, to the plurality of edges, each edge encoding indicating one or more attributes of the respective edge,
the class data include one or more class encodings representing one or more respective classes of the biological system, each class encoding indicating one or more attributes of the respective class of the biological system, and
the training sample data comprise a plurality of records derived from a respective plurality of second samples of the biological system, each record indicating presence,
absence, and/or expression levels of one or more of the plurality of entities in the
respective second sample of the biological system; and
determining one or more attributes of the first sample of the biological system based on output of the machine learning model.

80. The method of claim 79, wherein determining one or more attributes of the first sample of the biological system comprises determining presence, absence, and/or expression levels of one or more of the plurality of entities in the first sample of the biological system.

81. The method of claim 79, wherein determining one or more attributes of the first sample of the biological system comprises classifying the first sample as healthy or diseased based on the determined presence, absence, and/or expression levels of one or more of the plurality of entities in the first sample of the biological system.

82. The method of claim 79, wherein determining one or more attributes of the first sample of the biological system comprises determining one or more mechanisms of action in the first sample of the biological system, and/or determining one or more pharmacokinetic and/or pharmacodynamic properties of the first sample of the biological system.

83. The method of claim 79, wherein determining one or more attributes of the first sample of the biological system comprises determining one or more second classes to which the first sample of the biological system belongs.

84. The method of claim 79, wherein determining one or more attributes of the first sample of the biological system comprises determining a presence of cytotoxicity, growth inhibition, and/or apoptosis in the first sample of the biological system.

85. The method of claim 79, wherein the graph is a bond graph.

86. The method of claim 79, wherein the plurality of entities include one or more proteins, one or more genes, one or more transcripts, one or more small molecules, one or more biomolecular complexes, and/or one or more regulators.

87. The method of claim 79, wherein the plurality of interactions include one or more biochemical reactions, one or more transcription events, one or more translation events, one or more physical regulations, one or more indirect regulations, one or more degradations, one or more genomic connections, and/or one or more pathways.

88-111. (canceled)

112. The method of claim 79, wherein the one or more classes of the biological system include a tissue or cell type of the biological system, and wherein the one or more class encodings include a tissue type encoding representing the tissue type of the biological system.

113-115. (canceled)

116. The method of claim 79, wherein the one or more classes of the biological system include a disease type of the biological system, and wherein the one or more class encodings include a disease type encoding representing the disease type of the biological system.

117-119. (canceled)

120. The method of claim 79, wherein the one or more classes of the biological system include a therapeutic agent applied to the biological system, and wherein the one or more class encodings include a therapeutic agent encoding representing the therapeutic agent applied to the biological system.

121-130. (canceled)

131. A computer system for generating a model of a biological system, the computer system comprising:
one or more processing devices; and
one or more memory devices storing instructions which, when executed by the one or more processing devices, cause the computer system to perform the method of claim 1.

132. A computer system for modeling a biological system, comprising:
one or more processing devices; and
one or more memory devices storing instructions which, when executed by the one or more processing devices, cause the computer system to perform the method of claim 79.

133. A computer readable storage medium storing instructions that are configured, when executed by one or more computers, to cause the one or more computers to perform the method of claim 1.

134. A computer readable storage medium storing instructions that are configured, when executed by one or more computers, to cause the one or more computers to perform the method of claim 79.
